(19) United States
(12) Patent Application Publication
(10) Pub. No.: US 2018/0078773 A1
(43) Pub. Date: Mar. 22, 2018

(54) MULTI-DEVICE CARDIAC RESYNCHRONIZATION THERAPY WITH MODE SWITCHING TIMING REFERENCE

(71) Applicant: CARDIAC PACEMAKERS, INC., ST. PAUL, MN (US)

(72) Inventors: Pramodsingh Hirasingh Thakur, Woodbury, MN (US); Qi An, Blaine, MN (US); Krzysztof Z. Siejko, Maple Grove, MN (US); Yinghong Yu, Shoreview, MN (US); Stephen J. Hahn, Shoreview, MN (US)

(73) Assignee: CARDIAC PACEMAKERS, INC., ST. PAUL, MN (US)

(21) Appl. No.: 15/710,118

(22) Filed: Sep. 20, 2017

Related U.S. Application Data
(60) Provisional application No. 62/397,635, filed on Sep. 21, 2016.

(51) Int. Cl. A61N 1/37; A61N 1/368; A61N 1/39

(52) U.S. Cl. CPC A61N 1/3706; A61N 1/37288; A61N 1/39622; A61N 1/368

(57) ABSTRACT

Methods, systems and devices for providing cardiac resynchronization therapy (CRT) to a patient using a leadless cardiac pacemaker (LCP) and an extracardiac device (ED). The system is configured to have available for use a plurality of modes for managing the timing of the CRT pacing delivery by the LCP acting in cooperation with the ED. The system is further configured to use various metrics to determine whether and when to switch from one of the CRT timing modes to another of the timing modes.

### CROSS REFERENCE TO RELATED APPLICATIONS

The present application claims the benefit of and priority to U.S. Provisional Patent Application Ser. No. 62/397,635, filed Sep. 21, 2016, the disclosure of which is incorporated herein by reference.

### BACKGROUND

Cardiac resynchronization therapy (CRT) modifies the electrical activation and contractions of the heart's chambers to enhance pumping efficiency. Benefits may include increased exercise capacity and reduced hospitalization and mortality. More particularly, CRT devices operate by affecting the timing of contraction of one or more cardiac chambers relative to one or more other cardiac chambers. For example, contractions of one or more of the ventricle(s) may be timed relative to contraction of the atria, or contractions of the left and right ventricles may be timed relative to one another.

A “fusion” beat occurs when multiple activation signals affect the same cardiac tissue at the same time. For example, electrical fusion between pacing of one ventricle with spontaneous activation of another ventricle (for example, paced left ventricular (LV) activation and intrinsic right ventricular (RV) activation) produces a fusion beat. The generation of fusion beats is a goal of CRT in many circumstances.

Prior systems generally include intracardiac electrodes coupled via transvenous leads to an implanted pulse generator. The leads of such systems are widely known as introducing various morbidities and are prone to eventual conductor and/or insulator failure. Such issues likely reduce usage of CRT within the indicated population of heart failure patients.

Such prior lead systems typically include ventricular and atrial components to facilitate sensing of atrial and ventricular events to enhance CRT timing. For example, in some patients, CRT may be achieved by pacing the left ventricle at a specific time relative to detection of an atrial event. The atrial signal may conduct to the right ventricle (RV) via natural conduction to generate an RV contraction, with paced LV contraction occurring at a desirable time relative to the RV contraction to yield a fusion beat. The interval from the atrial sensed event to the LV pace may be adjusted to enhance cardiac response in prior systems.

Newer generation pacemakers include the leadless cardiac pacemaker (LCP), which can be implanted entirely within the heart and does not require a transvenous (or any) lead. Such devices are commercially available on a limited basis, but are currently indicated for and capable of use in only bradycardia pacing. With further enhancements, the LCP also presents an opportunity to provide an alternative to traditional CRT using transvenous leads. New and alternative systems, devices and methods directed at providing CRT using the LCP are desired.

### Overview

The present inventors have recognized, among other things, that a problem to be solved is that the absence of an intracardiac lead makes detection of an atrial event for purposes of CRT potentially difficult for a system using one or more ventricular LCP devices. A second implantable device, such as a subcutaneous cardiac monitor (SCM), a subcutaneous implantable cardiac defibrillator (SICD), or a substernal variant of the SICD, may be used to assist in the timing of delivery of LCP CRT therapy pacing. Such a second device may in some examples be referred to as an extracardiac device. There are various ways the second device can assist with achieved desired timing of the CRT therapy, but each has shortcomings.

In some examples, the second device may be configured to sense and detect atrial events, such as the P-wave, to trigger LCP pacing within the same cardiac cycle as the detected atrial events. Sensing the P-wave from an extracardiac device may be difficult if, for example, the P-wave is variable or sensed signals are noisy.

In some examples, the second device may be configured to sense and detect other events such as a septal event, for example, the onset of a Q-wave, to trigger LCP pacing within the same cardiac cycle as the detected atrial events. This approach may require very tight timing, as the pace therapy must occur very quickly after Q-wave onset; to achieve the very tight timing may require a very sensitive Q-wave detector that may be susceptible to noise.

Some examples may be predictive in that a retrospective analysis is performed on one or more prior CRT-paced cardiac cycles to determine if desired timing or outcome has occurred, and adjustments are made to pace timing to change timing of future therapy delivery, thus predicting when native cardiac events will occur based on knowledge from prior cardiac cycles and using the prediction to tailor pacing pulse timing. In some “predictive” examples, the second device may sense signals before and/or after a pacing pulse is delivered by the LCP to determine one or more metrics related to the timing of the delivered therapy; if desired timing was not achieved, timing of the pace delivery may be adjusted for future therapy delivery. In other predictive examples, the second device may sense the evoked response to pace therapy delivery in order to determine whether a desired cardiac outcome, such as a fusion beat, has been attained; if the desired cardiac outcome does not occur, timing of the pace delivery may be adjusted for future therapy delivery. These predictive models may not track quickly to changes in the underlying rhythm and can get out of sync with cardiac activity.

The present inventors have recognized in light of the above that methods and devices to change the mode of pace timing calculation provide new and useful alternatives to facilitate the multi-device CRT that is envisioned.

A first non-limiting example takes the form of an implantable medical device (IMD) configured for use as part of a cardiac therapy system comprising a leadless cardiac pacemaker (LCP) for delivering cardiac resynchronization therapy (CRT) and the IMD, the IMD comprising: a plurality of electrodes for sensing cardiac signals; communication circuitry for communicating with the LCP; and operational circuitry configured to receive sensed cardiac signals from the plurality of electrodes and analyze cardiac activity. In the first non-limiting example, the operational circuitry is configured to selectively implement a first mode of CRT pacing using first criteria for timing the delivery of CRT pacing by the LCP; the operational circuitry is configured to selectively implement a second mode of CRT pacing using second criteria for timing the delivery of CRT pacing by the LCP; and the operational circuitry is configured to select a mode for implementation amongst at least the first and second modes of CRT pacing and communicate to the LCP to implement the selected mode.

Additionally or alternatively, the operational circuitry may be configured to assess reliability of the at least first and second modes by analyzing data related to the first and second criteria, and to use the reliability to select a mode for implementation.

Additionally or alternatively, the operational circuitry may be configured to assess quality of a selected one of the at least first and second modes of CRT pacing to determine whether the selected one of the at least first and second modes is effectively providing CRT, and to use the quality to select a mode for implementation.

Additionally or alternatively, the quality may be assessed using analysis to determine whether a fusion beat has occurred.

Additionally or alternatively, the operational circuitry may be configured to assess quality of a selected one of the at least first and second modes of CRT pacing to determine whether the selected one of the at least first and second modes will likely effectively provide CRT, and to use the quality to select a mode of CRT pacing for implementation.

Additionally or alternatively, the operational circuitry may be configured to operate at least the first and second modes of CRT pacing to determine timing of pacing outputs that the first and second modes would have generated for a plurality of cardiac cycles, and to compare to actual timing of pace delivery by the LCP to assess past accuracy of the at least first and second modes of CRT pacing.

Additionally or alternatively, the operational circuitry may be configured to generate one or more measures of probability related to the past accuracy of the at least first and second modes of operation.

Additionally or alternatively, the operational circuitry may be configured to assess reliability of the at least first and second modes by analyzing data related to the first and second criteria, and to use the reliability to select a mode for implementation.

A second non-limiting example takes the form of an implantable medical device (IMD) configured for use as part of a cardiac therapy system comprising a leadless cardiac pacemaker (LCP) for delivering cardiac resynchronization therapy (CRT) and the IMD, the IMD comprising: a plurality of electrodes for sensing cardiac signals; communication circuitry for communicating with the LCP; and operational circuitry configured to receive sensed cardiac signals from the plurality of electrodes and analyze cardiac activity; wherein the operational circuitry is configured to selectively implement a first mode of CRT pacing using first criteria for timing the delivery of CRT pacing by the LCP. Further in the second non-limiting example, the operational circuitry is configured to selectively implement a second mode of CRT pacing using second criteria for timing the delivery of CRT paces by the LCP; and the operational circuitry is configured cooperatively use the first and second modes of CRT pacing by: analyzing data to generate first pace timing information using the first mode of CRT pacing for a set of cardiac cycles; analyzing data to generate second pace timing information using the second mode of CRT pacing for the set of cardiac cycles; make one or more adjustments as follows: adjust the first mode of CRT pacing using the second pace timing information; or adjust the second mode of CRT pacing using the first pace timing information.

A third non-limiting example, takes the form of an implantable medical device (IMD) configured for use as part of a cardiac therapy system comprising a leadless cardiac pacemaker (LCP) for delivering cardiac resynchronization therapy (CRT) and the IMD, the IMD comprising: a plurality of electrodes for sensing cardiac signals; communication circuitry for communicating with the LCP; and operational circuitry configured to receive sensed cardiac signals from the plurality of electrodes and analyze cardiac activity; wherein the operational circuitry is configured to selectively implement a first mode of CRT pacing using first criteria for timing the delivery of CRT pacing by the LCP; wherein the operational circuitry is configured to selectively implement a second mode of CRT pacing using second criteria for timing the delivery of CRT paces by the LCP; and wherein the operational circuitry is configured cooperatively use the first and second modes of CRT pacing by: analyzing data to generate first pace timing information using the first mode of CRT pacing for a set of cardiac cycles; analyzing data to generate second pace timing information using the second mode of CRT pacing for the set of cardiac cycles; take action to cause CRT pacing using at least one of the first pace timing information and the second pace timing information for the set of cardiac cycles; and store data for each of the first mode and the second mode to allow analysis of the probability that either of the first or second modes would cause fusion beats.

A fourth non-limiting example takes the form of an implantable medical device (IMD) configured for use as part of a cardiac therapy system comprising a leadless cardiac pacemaker (LCP) for delivering cardiac resynchronization therapy (CRT) and the IMD, the IMD comprising: a plurality of electrodes for sensing cardiac signals; communication circuitry for communicating with the LCP; and operational circuitry configured to receive sensed cardiac signals from the plurality of electrodes and analyze cardiac activity; wherein the operational circuitry is configured to selectively implement a first mode of CRT pacing using first criteria for timing the delivery of CRT pacing by the LCP; wherein the operational circuitry is configured to selectively implement a second mode of CRT pacing using second criteria for timing the delivery of CRT paces by the LCP; and wherein the operational circuitry is configured to: determine probabilities of pacing for CRT at a desirable time for at least the first and second modes of CRT pacing; determine a current reliability for each of the first and second modes of CRT pacing; select between the first and second modes of CRT pacing using the probabilities and the current reliabilities; and implement the selected mode of CRT pacing.

Additionally or alternatively, in any of the first to fourth non-limiting examples, the first mode of CRT pacing may use atrial electrical events as first criteria, and reliability of the first mode may be analyzed by observing one or more of amplitude, shape, or timing of the atrial electrical events.

Additionally or alternatively, in any of the first to fourth non-limiting examples, the first mode of CRT pacing may use atrial mechanical events as first criteria, and reliability of the first mode may then be analyzed by observing one or more of amplitude, shape, or timing of the atrial mechanical events.

Additionally or alternatively, in any of the first to fourth non-limiting examples, the first mode of CRT pacing may use septal electrical events as first criteria, and reliability of the first mode may be analyzed by observing one or more of amplitude, shape, or timing of the septal electrical events.

Additionally or alternatively, in any of the first to fourth non-limiting examples, the first mode of CRT pacing may use retrospective analysis of QRS complex shape as first criteria, and reliability of the first mode may be analyzed by observing one or more of amplitude, shape, or timing of QRS complexes.

Additionally or alternatively, in any of the first to fourth non-limiting examples, the first mode of CRT pacing may use retrospective analysis of a plurality of electrical events in the cardiac cycle as first criteria, and reliability of the first mode may be analyzed by observing one or more of amplitude, shape, or timing of the plurality of electrical events.

Additionally or alternatively, in any of the first to fourth non-limiting examples, the first mode of CRT pacing may use an optical signal indicating blood oxygenation or a volume signal indicating a ventricular volume as first criteria, and reliability of the first mode may be analyzed by observing one or more of amplitude, shape, timing, and relative changes in the first criteria.

Additionally or alternatively, in any of the first to fourth non-limiting examples, the second mode of CRT pacing may use atrial electrical events as second criteria, and reliability of the second mode may be analyzed by observing one or more of amplitude, shape, or timing of the atrial electrical events.

Additionally or alternatively, in any of the first to fourth non-limiting examples, the second mode of CRT pacing may use atrial mechanical events as second criteria, and reliability of the second mode may be analyzed by observing one or more of amplitude, shape, or timing of the atrial mechanical events.

Additionally or alternatively, in any of the first to fourth non-limiting examples, the second mode of CRT pacing may use septal electrical events as second criteria, and reliability of the second mode may be analyzed by observing one or more of amplitude, shape, or timing of the septal electrical events.

Additionally or alternatively, in any of the first to fourth non-limiting examples, the second mode of CRT pacing may use retrospective analysis of QRS complex shape as second criteria, and reliability of the second mode may be analyzed by observing one or more of amplitude, shape, or timing of QRS complexes.

Additionally or alternatively, in any of the first to fourth non-limiting examples, the second mode of CRT pacing may use retrospective analysis of a plurality of electrical events in the cardiac cycle as second criteria, and reliability of the second mode may be analyzed by observing one or more of amplitude, shape, or timing of the plurality of electrical events.

Additionally or alternatively, in any of the first to fourth non-limiting examples, the second mode of CRT pacing may use an optical signal indicating blood oxygenation or a volume signal indicating a ventricular volume as second criteria, and reliability of the second mode may be analyzed by observing one or more of amplitude, shape, timing, and relative changes in the second criteria.

Additionally or alternatively, in any of the first to fourth non-limiting examples, the IMD may take the form of a subcutaneous implantable defibrillator comprising therapy delivery circuitry for delivering cardiac electrical therapy. Additionally or alternatively, in any of the first to fourth non-limiting examples, the IMD may take the form of a subcutaneous cardiac monitor. Additionally or alternatively, in any of the first to fourth non-limiting examples, the IMD may take the form of a wearable cardiac monitoring device.

A fifth non-limiting example takes the form of a system comprising an IMD as in any of the first to fourth non-limiting examples, and an LCP configured to operate cooperatively with the IMB.

A sixth non-limiting example takes the form of a method of treating a patient comprising providing cardiac resynchronization therapy using: an IMB as recited in any of the first to fourth non-limiting examples, and an LCP configured to cooperatively operate with the IMD.

A seventh non-limiting example takes the form of a method of treating a patient comprising implanting an IMB as in any of the first to fourth non-limiting examples, implanting an LCP, and providing cardiac resynchronization therapy using the IMD and the LCP.

An eighth non-limiting example takes the form of a method of treating a patient comprising using an IMB as in any of the first to fourth non-limiting examples to manage cardiac resynchronization therapy delivered by an LCP.

A ninth non-limiting example takes the form of an implantable cardiac therapy system comprising: a leadless cardiac pacemaker (LCP) for delivering cardiac resynchronization therapy (CRT); and an implantable medical device (IMD) comprising a plurality of electrodes for sensing cardiac signals, communication circuitry for communicating with the LCP, and operational circuitry configured to receive sensed cardiac signals from the plurality of electrodes and analyze cardiac activity; wherein the operational circuitry is configured to selectively implement a first mode of CRT pacing using first criteria for timing the delivery of CRT pacing by the LCP; wherein the LCP is configured to selectively implement a second mode of CRT pacing using second criteria for timing the delivery of CRT paces by the LCP; and wherein the operational circuitry is configured to select a mode for implementation amongst at least the first and second modes of CRT pacing and communicate to the LCP to implement the selected mode.

Additionally or alternatively in the ninth non-limiting example, the first mode of CRT pacing may use atrial electrical events as first criteria, and reliability of the first mode may be analyzed by observing one or more of amplitude, shape, or timing of the atrial electrical events.

Additionally or alternatively in the ninth non-limiting example, the first mode of CRT pacing may use atrial mechanical events as first criteria, and reliability of the first mode is analyzed by observing one or more of amplitude, shape, or timing of the atrial mechanical events.

Additionally or alternatively in the ninth non-limiting example, the first mode of CRT pacing may use septal electrical events as first criteria, and reliability of the first mode may be analyzed by observing one or more of amplitude, shape, or timing of the septal electrical events.

Additionally or alternatively in the ninth non-limiting example, the first mode of CRT pacing may use retrospective analysis of QRS complex shape as first criteria, and reliability of the first mode may be analyzed by observing one or more of amplitude, shape, or timing of QRS complexes.

Additionally or alternatively in the ninth non-limiting example, the first mode of CRT pacing may use retrospective analysis of a plurality of electrical events in the cardiac cycle as first criteria, and reliability of the first mode may be analyzed by observing one or more of amplitude, shape, or timing of the plurality of electrical events.

Additionally or alternatively in the ninth non-limiting example, the first mode of CRT pacing may use an optical signal indicating blood oxygenation or a volume signal indicating a ventricular volume as first criteria, and reliability of the first mode may be analyzed by observing one or more of amplitude, shape, timing, and relative changes in the first criteria.

Additionally or alternatively in the ninth non-limiting example, the operational circuitry is configured to select the first mode of CRT pacing by default, and to switch to the second mode of CRT pacing in response to a failure of the first mode of CRT pacing to generate fusion beats.

Additionally or alternatively in the ninth non-limiting example, the operational circuitry is configured to select the first mode of CRT pacing by default, and to switch to the second mode of CRT pacing in response to finding that the first mode of CRT pacing is unreliable due to changes in or absence of a signal relied upon by the first mode of CRT pacing.

A tenth non-limiting example takes the form of a method of treating a patient comprising using an system as in the ninth non-limiting example to provide cardiac resynchronization therapy to the patient.

An eleventh non-limiting example takes the form of a method of treating a patient comprising implanting a system as in the ninth non-limiting example, and providing cardiac resynchronization therapy using the system.

This overview is intended to provide an introduction to the subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a patient having a plurality of implantable medical devices;

FIG. 2 shows an illustrative implantable medical device;

FIG. 3 shows an illustrative implantable leadless cardiac pacemaker;

FIG. 4 shows an overall method of use of a system;

FIGS. 5-12 show a number of illustrative approaches to pacing and pace timing for a multi-device implantable system;

FIG. 13 illustrates in block flow form an example for pacing and switching among a plurality of pacing modes;

FIG. 14 illustrates mode switching among a plurality of pacing modes;

FIGS. 15-16 show illustrative examples of selecting a pacing mode;

FIGS. 17-22 show in block flow form a number of examples for assessing pacing mode reliability;

FIGS. 23-24 show illustrative examples for assessing quality of delivered pace therapy;

FIG. 25 shows an illustrative example using posterior probability;

FIG. 26 shows an example incorporating both posterior probability and mode reliability; and

FIG. 27 shows another illustrative example.

### DETAILED DESCRIPTION

The following description should be read with reference to the drawings. The description and the drawings, which are not necessarily to scale, depict illustrative embodiments and are not intended to limit the scope of the disclosure.

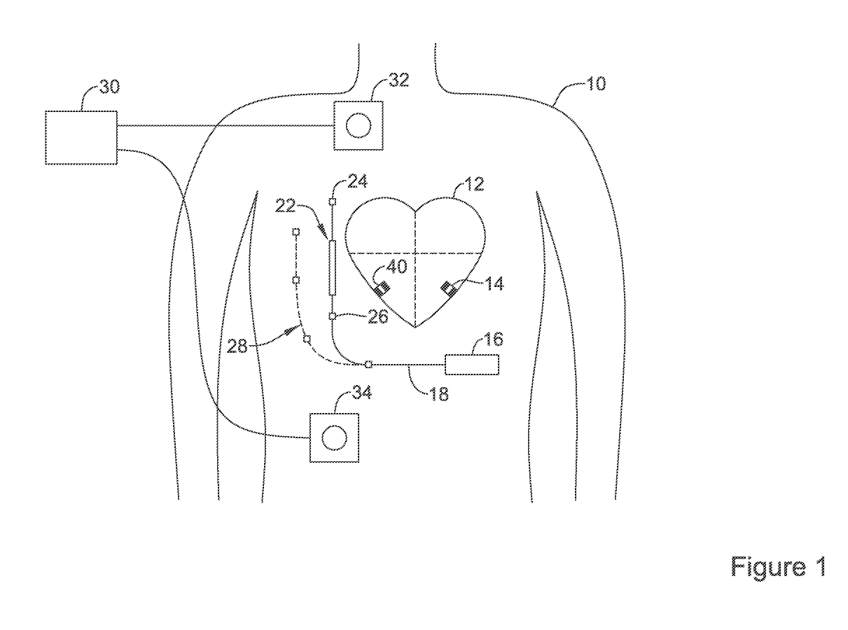

FIG. 1 illustrates a patient 10 with a first implanted medical device, shown as a leadless cardiac pacemaker (LCP) 14 implanted inside the heart 12, in the left ventricle for illustrative purposes. The LCP 14 may be implanted in other chambers, such as the right ventricle or in the atrium, and more than one LCP may be provided.

A second medical device in the form of a subcutaneous implantable defibrillator (SICD) having a left axillary canister 16 and a lead 18 is also present. The illustrative lead 18 is shown with a defibrillation coil 22 and sensing electrodes 24, 26 distal and proximal of the coil 22. The lead 18 may optionally include a bifurcation 28 to provide an additional set of sensing or stimulus providing electrodes, if desired.

In some embodiments the lead may be as shown, for example, in U.S. Pat. No. 9,079,035, titled ELECTRODE SPACING IN A SUBCUTANEOUS IMPLANTABLE CARDIAC STIMULUS DEVICE, the disclosure of which is incorporated herein by reference. Rather than bifurcation, plural leads may be provided as shown, for example, in U.S. Pat. No. 7,149,575, titled SUBCUTANEOUS CARDIAC STIMULATOR DEVICE HAVING AN ANTERIORLY POSITIONED ELECTRODE. Any suitable design for single, multiple, or bifurcated implantable leads may be used.

The lead 18 may be implanted entirely subcutaneously, such as by extending across the anterior or posterior of the chest, or by going partly across the chest in a lateral/medial direction and then superiorly toward the head along the sternum. Some examples and discussion of subcutaneous lead implantation may be found in U.S. Pat. No. 8,157,813, titled APPARATUS AND METHOD FOR SUBCUTANEOUS ELECTRODE INSERTION, and US PG Publication No. 20120029335, titled SUBCUTANEOUS LEADS AND METHODS OF IMPLANT AND EXPLANT, the disclosures of which are incorporated herein by reference. Additional subcutaneous placements are discussed in U.S. Pat. No. 6,721,597, titled SUBCUTANEOUS ONLY IMPLANTABLE CARDIOVERTER DEFIBRILLATOR AND OPTIONAL PACER, and the above mentioned U.S. Pat. No. 7,149,575, the disclosures of which are incorporated herein by reference.

A substernal placement may be used instead, with one finger 18/20 or the entire distal end of the lead (that is, the end distant from the canister 16) going beneath the sternum. Some examples of such placement are described in US PG Patent Pub. No. 2017/0021159, titled SUBSTERNAL PLACEMENT OF A PACING OR DEFIBRILLATING ELECTRODE, the disclosure of which is incorporated herein by reference. Still another alternative placement is shown in U.S. Provisional Patent Application No. 62/371,343, titled IMPLANTATION OF AN ACTIVE MEDICAL DEVICE USING THE INTERNAL THORACIC VASCULATURE, the disclosure of which is incorporated herein by reference.

The devices 14 and 16 may communicate with one another and/or with an external programmer 30 using conducted communication, in some examples. Conducted communication is communication via electrical signals which propagate via patient tissue and are generated by more or less ordinary electrodes. By using the existing electrodes of the implantable devices, conducted communication does not rely on an antenna and an oscillator/resonant circuit having a tuned center frequency or frequencies common to both transmitter and receiver. RF or inductive communication may be used instead. Alternatively the devices 14 and 16 may communicate via inductive, optical, sonic, or RF communication, or any other suitable medium.

The programmer 30 may optionally use a wand (not shown) and/or skin electrodes 32 and 34 to facilitate communication. For example, skin electrodes 32 and 34 may be used for conducted communication with an implantable device. For other communication approaches such as RF or inductive communication, the programmer 30 may use a programming wand or may have an antenna integral with the programmer 30 housing for communication. Though not shown in detail, the programmer 30 may include any suitable user interface, including a screen, buttons, keyboard, touchscreen, speakers, and various other features widely known in the art.

Subcutaneous implantable defibrillators may include, for example, the Emblem S-ICD System™ offered by Boston Scientific Corporation. Combinations of subcutaneous defibrillators and LCP devices are discussed, for example, in US PG Patent Publication Nos. 20160059025, 20160059024, 20160059022, 20160059007, 20160038742, 20150297902, 20150196769, 20150196758, 20150196757, and 20150196756, the disclosures of which are incorporated herein by reference. The subcutaneous defibrillator and LCP may, for example, exchange data related to cardiac function or device status, and may operate together as a system to ensure appropriate determination of cardiac condition (such as whether or not a ventricular tachyarrhythmia is occurring), as well as to coordinate therapy such as by having the LCP deliver antitachycardia pacing in an attempt to convert certain arrhythmias before the subcutaneous defibrillator delivers a defibrillation shock.

In some examples, rather than a therapy device such as the SICD shown in FIG. 1, a second implantable medical device may take the form of an implantable monitoring device such as a subcutaneous cardiac monitor (SCM). An SCM may be, for example, a loop monitor that captures data under select conditions using two or more sensing electrodes on a housing thereof and/or attached thereto with a lead. Such monitors have found use to assist in diagnosing cardiac conditions that may be infrequent or intermittent, or which have non-specific symptoms. In the context of the present invention, an SCM, or even a wearable cardiac monitor, may be used in place of the SICD as described in any of the following examples.

Several examples focus on using a left ventricular LCP 14. However, some examples may instead use a right ventricular LCP 40, and other examples may include both the left ventricular LCP 14 and right ventricular LCP 40. In other examples, a three implant system may include two LCP devices 14, 40, as well as a subcutaneous device such as the SICD 16. In still other examples, an atrial-placed LCP (not shown) may also be included or may take the place of one of the ventricular LCP devices 14, 40.

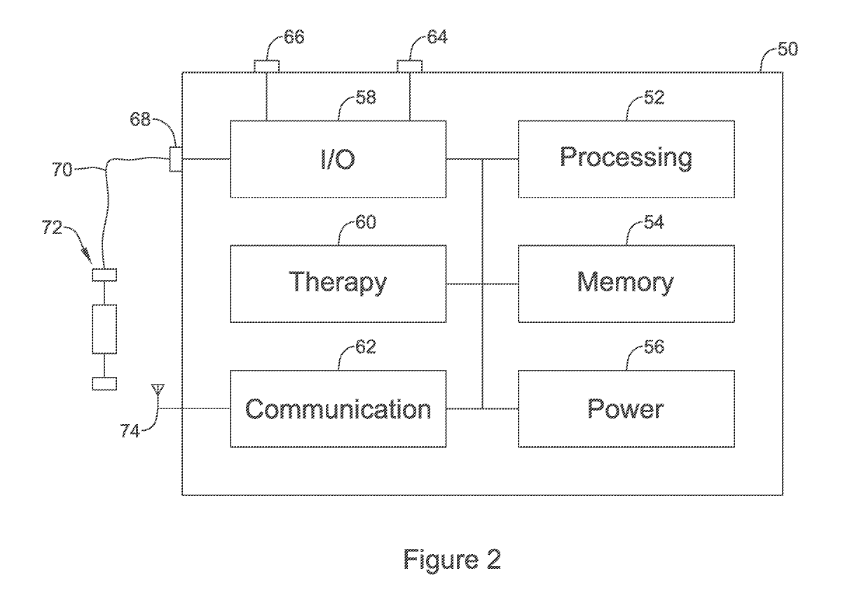

FIG. 2 illustrates a block diagram of an implantable medical device. The illustration indicates various functional blocks within a device 50, including a processing block 52, memory 54, power supply 56, input/output circuitry 58, therapy circuitry 60, and communication circuitry 62. These functional blocks make up the operational circuitry of the device. The I/O circuitry 58 can be coupled to one or more electrodes 64, 66 on the housing of the device 50, and may also couple to a header 68 for attachment to one or more leads 70 having additional electrodes 72.

The processing block 52 will generally control operations in the device 50 and may include a microprocessor or microcontroller and/or other circuitry and logic suitable to its purpose. A state machine may be included. Processing block 52 may include dedicated circuits or logic for device functions such as converting analog signals to digital data, processing digital signals, detecting events in a biological signal, etc. The memory block may include RAM, ROM, flash and/or other memory circuits for storing device parameters, programming code, and data related to the use, status, and history of the device 50. The power supply 56 typically includes one to several batteries, which may or may not be rechargeable depending on the device 50. For rechargeable systems there would additionally be charging circuitry for the battery (not shown).

The I/O circuitry 58 may include various switches or multiplexors for selecting inputs and outputs for use. I/O circuitry 58 may also include filtering circuitry and amplifiers for pre-processing input signals. In some applications the I/O circuitry will include an H-Bridge to facilitate high power outputs, though other circuit designs may also be used. Therapy block 60 may include capacitors and charging circuits, modulators, and frequency generators for providing electrical outputs. A monitoring device may omit the therapy block 60 and may have a simplified I/O circuitry used simply to capture electrical or other signals such as chemical or motion signals.

The communication circuitry 62 may be coupled to an antenna 74 for radio communication (such as Medradio, ISM, Bluetooth, or other RF), or alternatively to a coil for inductive communication, and/or may couple via the I/O circuitry 58 to a combination of electrodes 64, 66, 72, for conducted communication. Communication circuitry 62 may include a frequency generator/oscillator and mixer for creating output signals to transmit via the antenna 74. Some devices 50 may include a separate or even off-the shelf ASIC for the communications circuitry 62, for example. For devices using an inductive communication output, an inductive coil may be included. Devices may use optical or acoustic communication, and suitable circuits, transducers, generators and receivers may be included for these modes of communication as well or instead of those discussed above.

As those skilled in the art will understand, additional circuits may be provided beyond those shown in FIG. 2. For example, some devices 50 may include a Reed switch, Hall Effect device, or other magnetically reactive element to facilitate magnet wakeup, reset, or therapy inhibition of the device by a user, or to enable an MRI protection mode. A device lacking a lead may have plural electrodes on the housing thereof, as indicated at 64, 66, but may omit the header 68 for coupling to lead 70. In one example, a leadless device may use a header to couple to an electrode support feature that is attached to or wraps around the device housing.

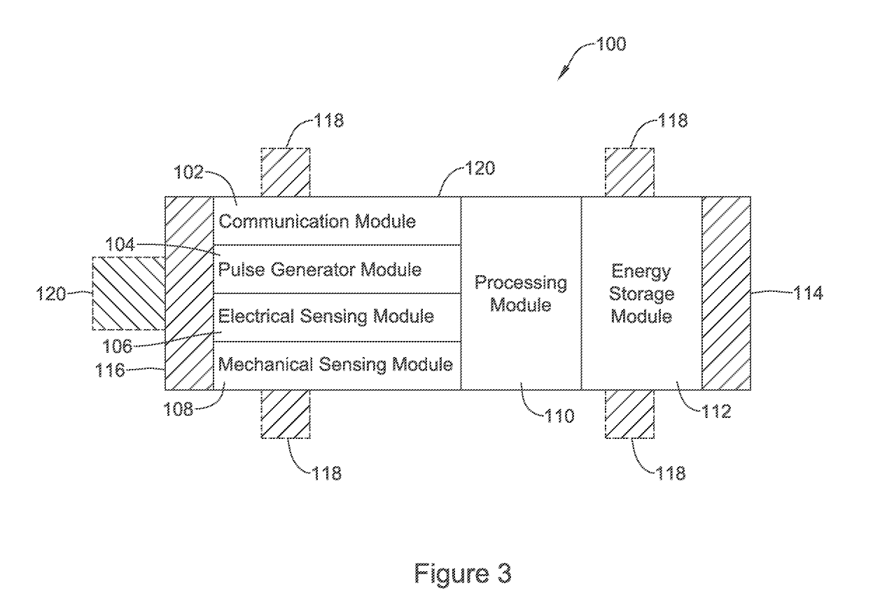

FIG. 3 shows an illustrative LCP design. The LCP 100 is shown as including several functional blocks including a communications module 102, a pulse generator module 104, an electrical sensing module 106, and a mechanical sensing module 108. A processing module 110 may receive data from and generate commands for outputs by the other modules 102, 104, 106, 108. An energy storage module is highlighted at 112 and may take the form of a rechargeable or non-rechargeable battery, or a supercapacitor, or any other suitable element. Various details of the internal circuitry, which may include a microprocessor or a state-machine architecture, are further discussed in US PG Patent Publications 20150360036, titled SYSTEMS AND METHODS FOR RATE RESPONSIVE PACING WITH A LEADLESS CARDIAC PACEMAKER, 20150224320, titled MULTI-CHAMBER LEADLESS PACEMAKER SYSTEM WITH INTER-DEVICE COMMUNICATION, 20160089539, titled REFRACTORY AND BLANKING INTERVALS IN THE CONTEXT OF MULTI-SITE LEFT VENTRICULAR PACING, and 20160059025, titled, MEDICAL DEVICE WITH TRIGGERED BLANKING PERIOD, as well as other patent publications. Illustrative architectures may also resemble those found in the Micra™ (Medtronic) or Nanostim™ (St. Jude Medical) leadless pacemakers.

The device is shown with a first end electrode at 114 and a second end electrode at 116. A number of tines 118 may extend from the device in several directions. The tines 118 maybe used to secure the device in place within a heart chamber. Another attachment structure is shown at 120 and may take the form of a helical screw, if desired. In some examples, tines 118 are used as the only attachment features. Tissue attachment and retrieval features may be included in the LCP including those features shown in US PG Patent Publications 20150051610, titled LEADLESS CARDIAC PACEMAKER AND RETRIEVAL DEVICE, and 20150025612, titled SYSTEM AND METHODS FOR CHRONIC FIXATION OF MEDICAL DEVICES, the disclosures of which are incorporated herein by reference. Fixation and retrieval structures may instead resemble that of the Micra™ (Medtronic) or Nanostim™ (St. Jude Medical) leadless pacemakers.

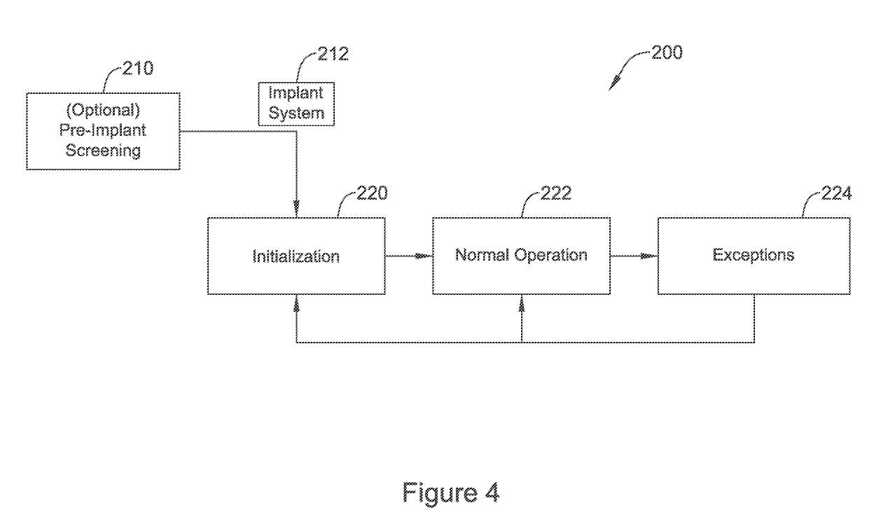

FIG. 4 shows an overall method of use of a system. The method 200 in this case goes back, optionally, to pre-implant screening, as indicated at 210. For example, the implantation of an SICD may occur following pre-implant screening for cardiac signal amplitude and/or signal to noise ratio, and/or to determine whether the patient's routine cardiac rhythm will be well managed using an SICD. Some example screening tools, metrics and methods discussed in U.S. Pat. No. 8,079,959, titled PATIENT SCREENING TOOLS FOR IMPLANTABLE CARDIAC STIMULUS SYSTEMS, and/or U.S. patent application Ser. No. 15/001,976, titled AUTOMATED SCREENING METHODS AND APPARATUSES FOR IMPLANTABLE MEDICAL DEVICES, the disclosures of which are incorporated herein by reference.

Pre-implant screening may also determine whether the patient is well suited to have a combined LCP/SICD or LCP/SCM system for CRT by assessing the presence or absence of a P-wave. P-wave related screening may be optional with the present invention, as various examples rely on SICD or SCM analysis of the QRS complex (or other cardiac signal) to confirm fusion, rather than the appearance or timing of the P-wave, to enhance or control CRT to attain desirable fusion.

The system(s) are then implanted at 212. Implantation may include the placement of an LCP on or in the heart, as well as placement of an SCM or SICD elsewhere in the patient such as between the ribs and the skin. The system may undergo intraoperative testing as is known in the art for each of LCP, SCM and SICD devices, to ensure adequate sensing configurations and/or therapy capability.

Next, the system undergoes initialization, at 220. Initialization may include, for example, the setting of various sensing and other parameters. Examples of initialization may include selecting of a sensing vector or combination of sensing vectors, such as in U.S. Pat. No. 7,783,340, titled SYSTEMS AND METHODS FOR SENSING VECTOR SELECTION IN AN IMPLANTABLE MEDICAL DEVICE USING A POLYNOMIAL APPROACH, and U.S. Pat. No. 8,483,843 SENSING VECTOR SELECTION IN A CARDIAC STIMULUS DEVICE WITH POSTURAL ASSESSMENT, the disclosures of which are incorporated herein by reference. Related concepts surrounding the use of multiple vector sensing are also disclosed in US PG Patent Pub. Nos. 2017/0112399, 2017/0113040, 2017/0113050, and 2017/0113053, the disclosures of which are incorporated herein by reference. Methods as discussed in US PG Patent Pub. No. 2017/0156617, titled AUTOMATIC DETERMINATION AND SELECTION OF FILTERING IN A CARDIAC RHYTHM MANAGEMENT DEVICE, the disclosure of which is incorporated herein by reference, may be used as well for setting filtering characteristics.

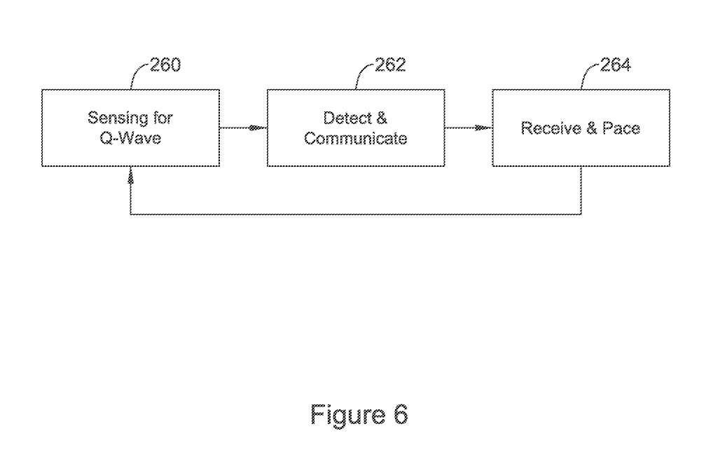

Initialization for an LCP may also include the setting of parameters for therapy including, for example, selecting pace shape, pulse width and/or amplitude. If plural LCPs are included in a system, the relative timing between pace deliveries amongst the plural LCPs, and other suitable features, may be set as well. Initialization may also include identifying a P-R interval for the patient, which can be done and used as discussed below relative to FIG. 6.

Once initialization 220 is completed, normal operation can occur as indicated at 222. Such operation may include CRT delivery in which a first device delivers pacing pulses for CRT purposes with the assistance of a second device such as an SICD or SCM.

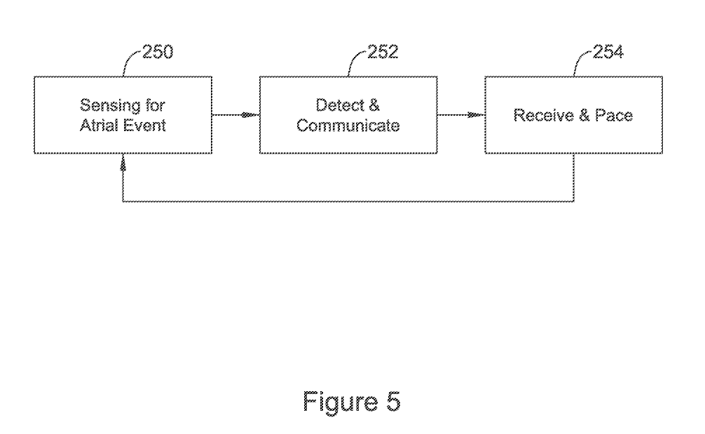

FIGS. 5-12 show a number of illustrative approaches to pacing and pace timing for CRT in a multi-device implantable system. Such approaches are referred to herein as modes of pacing for CRT. Different modes use different inputs or criteria for determining whether and/or when a pace therapy (a single pace impulse of a monophasic, biphasic, or other shape voltage or current controlled therapy output associated with a single cardiac cycle) is to be delivered. Any of the modes of pacing for CRT shown in FIGS. 5-12 may be used as a “normal operation” at 222 in FIG. 4, though different modes of pacing for CRT may have different initialization needs.

Multiple pace therapies, as used herein, means multiple individual paces, as opposed to pacing via different configurations or for different purposes. Thus the idea is to select between different modes of pacing to adaptably control the timing of CRT pacing therapy.

Some of the following examples call for each of an extracardiac device, such as an SICD or SCM to provide information or commands to an implanted LCP such as an LCP placed in the left ventricle of a patient. Some examples instead call for the LCP to perform its own assessments to perform pacing as needed. The aim in several examples is to provide effective CRT. Mode switching in various examples below may operate in systems capable of performing two or more of the illustrative modes in FIGS. 5-12, however, in keeping with the spirit of the present invention, other modes than those shown may be used in addition or instead.

FIG. 5 shows a mode in which an extracardiac device, such as an SICD or SCM, or a second implantable LCP, performs sensing for an atrial event 250, detects the atrial event and communicates to the LCP at 252. The LCP receive the communication and delivers pacing at 254. The LCP may be located in the left ventricle. The communication may take the form of a command to pace, or may instead simply provide information such as a notification that an atrial event has been sensed.

The atrial event may be an electrical signal detection, such as a P-wave, or likely P-wave, has been detected. See, for example, U.S. Provisional Patent Application Ser. No. 62/355,121, titled CARDIAC THERAPY SYSTEM USING SUBCUTANEOUSLY SENSED P-WAVES FOR RESYNCHRONIZATION PACING MANAGEMENT, the disclosure of which is incorporated herein by reference, for examples using a second device to detect an atrial electrical signal for use in CRT pacing. The atrial event may be a mechanical event instead, indicating atrial contraction. See, for example, U.S. Provisional Patent Application Ser. No. 62/359,055, titled METHOD AND SYSTEM FOR DETERMINING AN ATRIAL CONTRACTION TIMING FIDUCIAL IN A LEADLESS CARDIAC PACEMAKER SYSTEM, the disclosure of which is incorporated herein by reference, for examples of the LCP or a second device detecting an atrial mechanical signal for use in CRT pacing. For example, the S4 heart sound, which indicates atrial contraction may be detected and relied upon. In another example the A-wave, a pressure wave indicating atrial contraction, may be detected and relied upon.

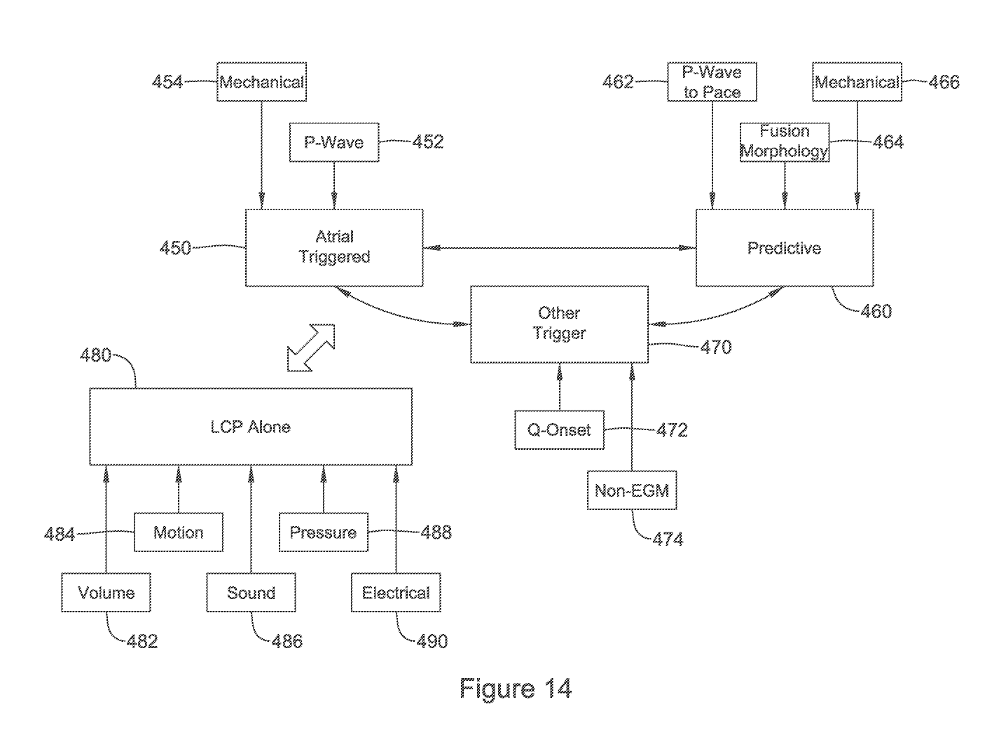

The electrical P-wave or other atrial event sensing may be difficult in some environment such as a noisy environment, or may be difficult in certain patients due to abnormal conduction, placement of sensing electrodes, etc. P-wave or other atrial event sensing may also be difficult if a patient has an atrial arrhythmia that prevents such sensing, for example, if a patient starts to experience atrial fibrillation. Patient movement and/or the patient's environment may affect the ability to sense a mechanical signal as well. Thus reliance on a single mode, while possibly useful in some patients all of the time, may not be enough in all patients all of the time. The same is true for many different approaches to pacing for CRT purposes when using an LCP. Further discussion of potential sources of difficulty with this and other modes will be briefly descried below with reference to FIG. 14.

FIG. 6 shows a mode in which an extracardiac device, such as an SICD or SCM, or a second implantable LCP, performs sensing for a ventricular/septal event such as the electrical Q-wave, as indicated at 260. When the Q-wave is detected at 262, a communication is issued to the LCP, which may be located in the left ventricle. The LCP receives the signal and delivers a pace therapy as indicated at 264. The communication may be informative, as in, a Q-wave has been identified or a Q-wave has been identified at a specific time, or may be a command to deliver pacing, depending on system configuration.

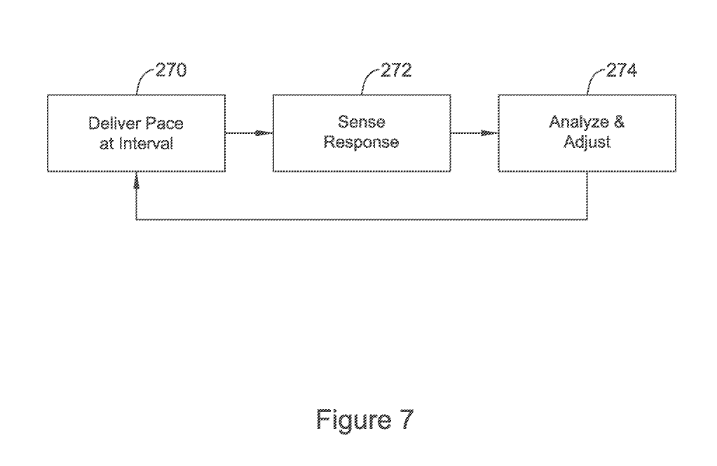

FIG. 7 shows a mode in which an LCP delivers pace therapy at a set interval, as indicated at 270. A second device, such as a second LCP or an extracardiac device such as an SICD or SCM, senses the response of the patients heart to the delivered pace therapy, as indicated at 272. The second device analyzes the signal it observed and determines whether an adjustment is needed, as indicated at 274, with any adjustments then being communicated.

For example, the sensing at 272 may include capturing the QRS complex following pace therapy at 270 and determining whether the QRS complex indicates a fusion beat has taken place. See U.S. Provisional Patent Application Ser. No. 62/378,866, titled CARDIAC RESYNCHRONIZATION USING FUSION PROMOTION FOR TIMING MANAGEMENT, the disclosure of which is incorporated by reference, for examples related to analysis of the QRS complex after pace therapy to identify fusion beats and modify pace timing as needed.

In another example, the sensing at 272 may include observing heart sounds and determining whether the cardiac response matches a desired outcome such as a fusion beat. For example, sensing 272 may observe a sequence of heart sound signals to indicate relative timing between valve events (closure, for example) indicating the timing of different chamber contractions to determine whether a desirable timing, pattern and/or sequence has occurred.

In another example, the sensing at 272 may use other physiologic measures to determine whether a pace therapy has done what is desired, such as by reference to pulse oxygenation, blood pressure signals, changes in cardiac volume, and/or cardiac motion. For example, sensing 272 may observe whether oxygenation peaks occur in desirable succession or at enhanced strengths, or whether blood pressure changes occur in a desirable sequence or timing or at a desired magnitude/strength. In other example, changes in cardiac volume and/or degrees of cardiac motion may be monitored to observe an improvement or change relative to baseline.

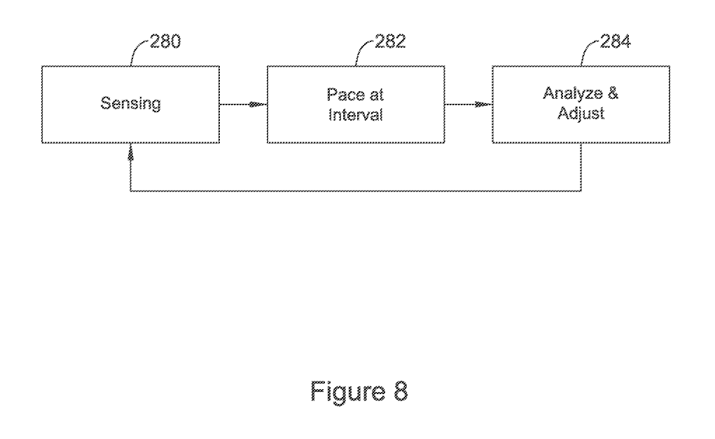

FIG. 8 shows another illustrative example in which sensing is performed by an extracardiac device such as an SICD or SCM or by a second LCP, as indicated at 280, across an interval of time during which a pace therapy is then delivered at 282 by an LCP which may be a left ventricular LCP. The sensed signal from block 280 is then analyzed and adjustments may then be made to the interval used for pace timing at block 282. For example, an interval from the pace therapy delivery to the R-wave, to the QRS complex, or from the P-wave to the pace therapy, may be calculated and/or assessed retrospectively to then make adjustments to tailor the desired timing. See U.S. Provisional Patent Application Ser. No. 62/378,880, titled INTEGRATED MULTI-DEVICE CARDIAC RESYNCHRONIZATION THERAPY USING P-WAVE TO PACE TIMING, the disclosure of which is incorporated herein by reference, for retrospective analysis of such features. Rather than an electrical signal analysis, the sensing at 280 may use mechanical sensors (an accelerometer, or pressure sensor for example) to find the timing of an atrial mechanical event.

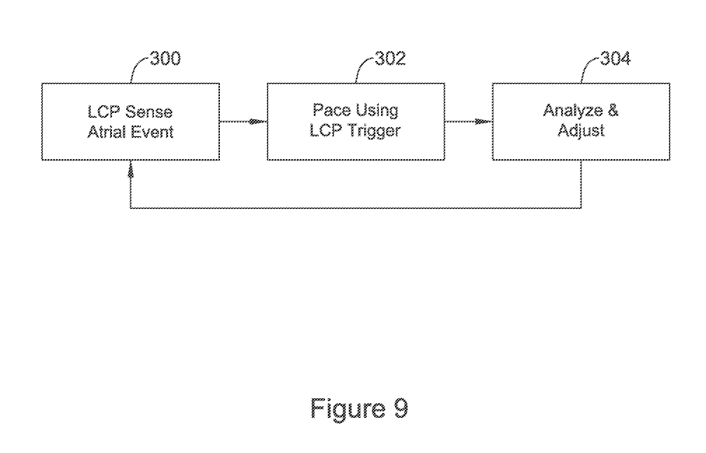

FIG. 9 shows another illustrative example of a pace timing mode. Here, an LCP senses an atrial event at 300 and delivers pacing using a trigger sensed by the LCP itself, as indicated at 302. A second device, such as a second LCP or an extracardiac device such as an SICD or SCM then analyzes the signals and calculates adjustments as indicated at 304. For example, the second device may reference an electrical signal (cardiac electrical signal), a mechanical signal (motion or pressure/sound sensor), or a physiological measure (pulse oxygenation) to perform analysis, and the analysis may include measuring outcomes (fusion morphology, or desired amount of motion or pressure change, volume change, or sounds) or parameters (the P-wave to Pace or Pace to R-wave duration, for example). In some alternative examples, the device that paces at 302 may also perform the analysis and adjustments at 304.

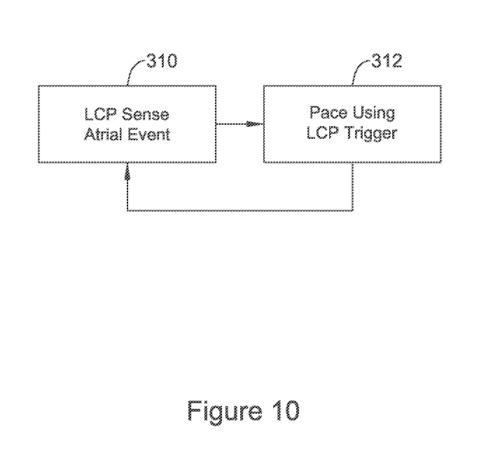

FIG. 10 shows an example in which the LCP senses an atrial event, either using mechanical or electrical signals, as indicated at 310. In response, the same LCP delivers a pace therapy at a time selected relative to the atrial event, as indicated at 312.

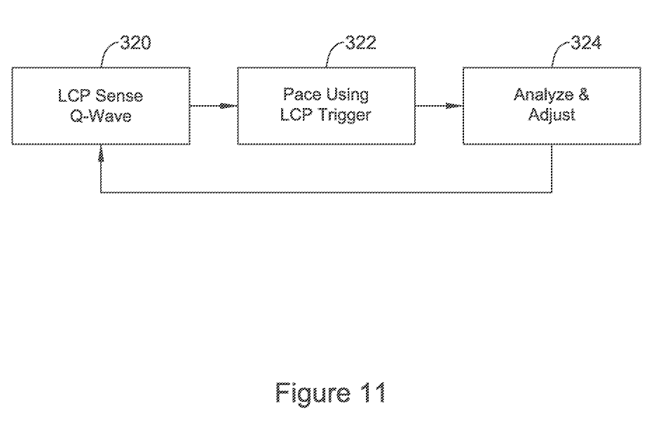

FIG. 11 shows an example in which the LCP senses a septal event, such as a Q-wave, as indicated at 320, and the same LCP then delivers pacing as shown at 322 based on the self-sensed septal event. A second device, such as a second LCP or an extracardiac device such as an SICD or SCM then analyzes the signals and calculates adjustments as indicated at 324. For example, the second device may reference an electrical signal (cardiac electrical signal), a mechanical signal (motion or pressure/sound sensor), or a physiological measure (pulse oxygenation) to perform analysis, and the analysis may include measuring outcomes (fusion morphology, or desired amount of motion or pressure change, volume change, or sounds) or parameters (the Q-wave to Pace interval for example). In some alternative examples, the device that paces at 322 may also perform the analysis and adjustments at 324.

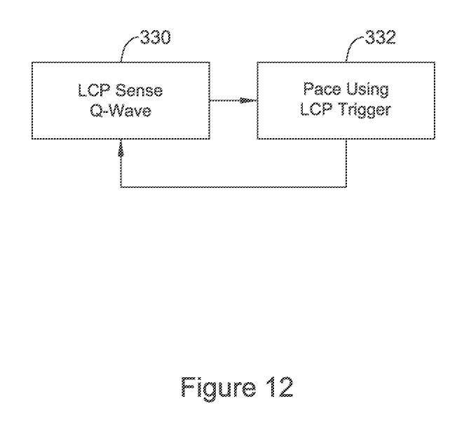

FIG. 12 shows an example in which the LCP senses a septal event, such as the Q-wave, as indicated at 330. In response, the same LCP delivers a pace therapy at a time selected relative to the septal event, as indicated at 332.

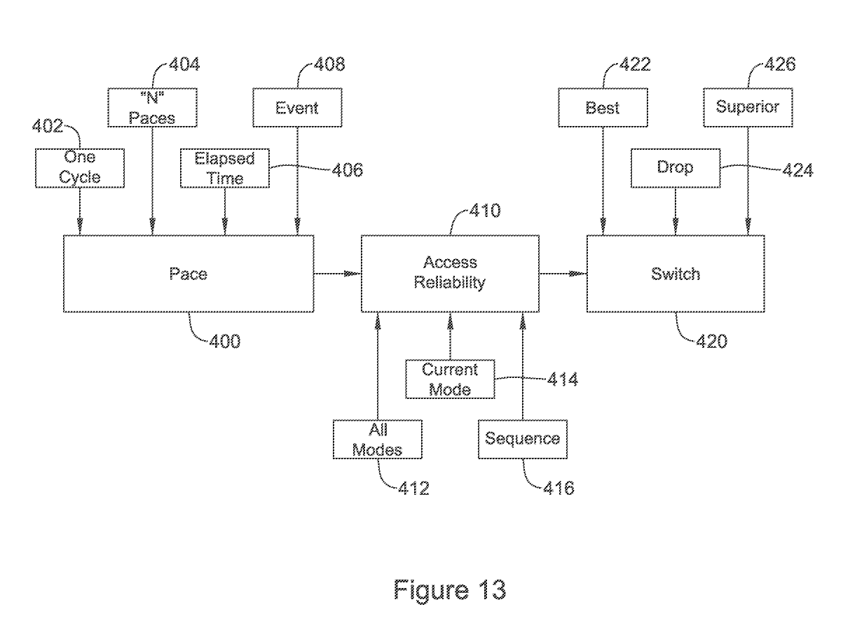

FIG. 13 shows an illustrative example for pacing and switching among a plurality of pacing modes. In the example method, one or more pace therapies are delivered at 400, and a system assesses the reliability of one or more modes of pace therapy timing control at 410. If needed, the pacing mode used to deliver pacing at 400 may be switched for a different mode at 420. The overall approach can take several related forms including, for example and without limitation, continuously switching after each pace therapy to a “best” mode as assessed using reliability 410 and/or other factors, (occasionally) switching from a current mode to a preferred or “best” mode upon determination that the current mode has encountered difficulties or upon some specified event occurring, or periodically reassessing, such as at a time interval or after a selected quantity of pace therapies are delivered, or otherwise, whether the presently used mode should be switched to a potentially better mode. Parameters used to determine which mode is “best” may vary; for example, there may be a preferred order of modes. The available modes may be ranked and analyzed in an order until an acceptable mode is found, rather than assessing several modes and selecting the best available.

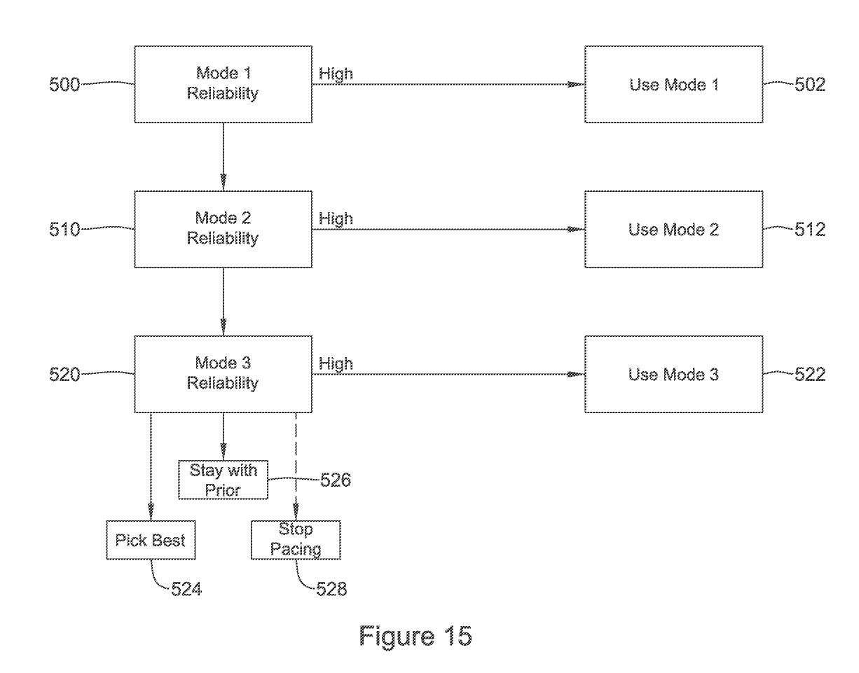

Going back through the detail of the example in FIG. 15, the assessment of reliability at 410 may occur after each cardiac cycle or pace therapy delivery 402, or may occur after a defined quantity of pace therapies are delivered at 404, or after a specified period of time has elapsed, as indicated at 406. In some examples, selected events may trigger assessment, as indicated at 408. For example and without limitation, an event triggering assessment may be a timeout of a mode of pacing therapy. Various events may be identified for purposes of triggering assessment at 408.

For example, a pacing mode as indicated above in FIG. 5 that relies on detection of an atrial event may timeout if no atrial events are detected for a period of time, for example, one or more cardiac cycles or one or more seconds of time. When the pacing mode cannot detect its targeted atrial events, it may rely on an exception handling mode to preserve a pace-to-pace interval (see, for example, U.S. Provisional Patent Application Ser. No. 62/355,121, titled CARDIAC THERAPY SYSTEM USING SUBCUTANEOUSLY SENSED P-WAVES FOR RESYNCHRONIZATION PACING MANAGEMENT, the disclosure of which is incorporated by reference, for certain examples. Repeated use of the exception handling mode may lead to timeout of the pacing mode. The timeout may indicate loss of the signal due to onset of an atrial arrhythmia, for example, or a change in patient posture affecting sense signal amplitude, or various other things.

In another example, a template may be used for retrospective analysis of the cardiac electrical response to identify fusion beats; if no fusion beats are sensed for a period of time or quantity of cardiac cycles (10 seconds or 10 cycles, or more or less, for example), this may be an event 408 triggering a reassessment of reliability. At a higher level, the failure to sense a signal that is relied upon, whether as a trigger or as verification of desired pace therapy outcome in CRT, may be deemed an event 408 that causes assessment of reliability at 410.

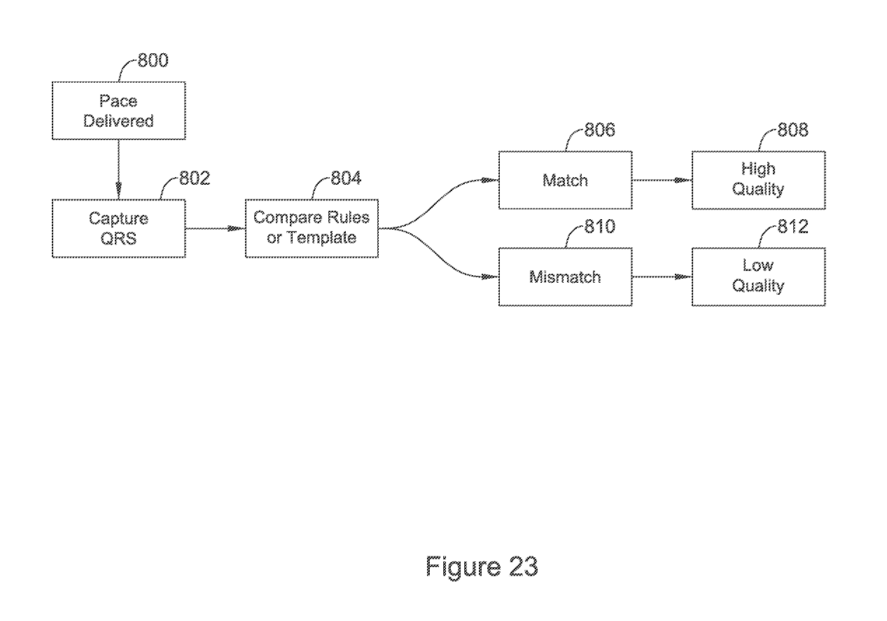

Turning to the reliability assessment 410, the method or device may look at all or a plurality of available pacing modes as indicated at 412. In another example, the method or device may review just the currently used mode as indicated at 414, and then assess other modes if difficulties are identified with the currently used mode. For example, a quality assessment as shown in FIG. 23 (or other examples), below, may be used to assess reliability of the current mode 414 by determining whether desirable pacing outcomes are occurring. In another example, the method or device may use a sequence of analysis of different modes from preferred to least preferred, as indicated at 416.

Finally, the decision to switch modes at 420 may take several forms. As indicated at 422, the “best” available mode may be selected, where best may indicate the mode deemed most reliable, or may be the first mode of a sequence of modes to be deemed adequate if a preset ranking or ordering of available modes is used.

In another example, a new mode may be selected only if the currently selected mode has dropped in quality or reliability as indicated at 424. A drop in quality may indicate that analysis of cardiac response to the pacing mode has not shown desirable outcomes—for example, fusion beats; a drop in reliability means that the signals on which a particular pacing mode relies are gone, losing amplitude or becoming inconsistent such as if an atrial electrical signal has dropped below a threshold amplitude or no longer matches a stored template.

In another example, superiority may be used as indicated at 426. Here, the system may stick with a current mode of pacing operation unless or until a superior mode is identified—that is, the reliability of the newly selected mode will have to overcome the reliability of the current mode by a margin or degree.

Some examples may allow mode switching to occur repeatedly. Other examples may invoke some quantity of hysteresis to prevent repeated switching such as by requiring a newly selected mode to remain in use for at least a minimum number of cardiac cycles/pace therapies, or period of time.

Upon selection of a new mode for CRT pacing, the method or system may, if needed, communicate amongst the devices. For example, an extracardiac device may communicate to an LCP and/or to a remote sensor used in the mode of pacing. In other examples, commanded pacing may be relied upon such that the LCP that delivers the CRT pacing does not need to know the basis of the commands it receives and no information about the mode selection is conveyed.

FIG. 14 illustrates mode switching among a plurality of pacing modes for CRT, with modes indicated at 450, 460, 470, and 480. Modes 450, 460, and 470 are each cooperative modes in which a left ventricular placed LCP delivers pace therapy and receives timing assistance from a second device such as an extracardiac device (SICD and/or SCM, for example) or a second LCP placed else wherein the heart, while mode 480 represents an independent mode of operation for the LCP, where the LCP itself determines pace timing for CRT.

For example, mode 450 is an atrial-triggered mode, which may use cardiac electrical information such as the P-wave, as indicated at 452. Alternatively, mechanical or other sensor information may be captured and used as a trigger, as indicated at 454, such as by identifying a heart sound, motion in the atrium, or pressure changes in the atrium or related to atrial activity.

Predictive mode 460 may operate by controlling a pace-to-pace interval and reviewing past result of pace therapy delivery to adjust the pace-to-pace interval based on a “prediction” of when will be the right time to deliver a next pace therapy. For example, a predictive mode may use analysis of prior P-wave to pace intervals, as indicated at 462, or may use a morphology assessment of a QRS complex to determine whether the QRS complex has a shape that indicates fusion, using for examples rules or templates in the analysis. In still further examples of predictive pacing 460, a mechanical signal, such as the timing of heart sounds in relative sequence, may be analyzed as indicated at 466 to optimize pace timing.

Other signals may be assessed as well, as indicated at 470, including the septal signal such as the Q-wave onset, as indicated at 472. Non-electrogram signals may be used, such as a heart sound emanating from other than the atria at 474.

An autonomous mode for CRT pacing by an LCP may be used as well, as indicated at 480. Such an LCP may be placed in the left ventricle, and may be capable of various analysis to help with triggered or predictive pacing management. For example, the LCP may monitor ventricular volume using an impedance measurement, as indicated at 482, triggering pacing when the volume reaches a threshold level or change. The LCP may detect motion, as indicated at 484 and trigger therapy. The LCP may have a sensor for sensing heart sounds and may detect a sound associated with atrial or right ventricular contraction, as indicated at 486. A pressure signal may monitored to detect changes indicating atrial or right ventricular contraction triggering therapy output. An electrical input 490 may be used by filtering to obtain a far-field signal from the atrium, or the LCP may have a short lead accessing the atria and can sense atrial signals. Any of these inputs 482, 484, 486, 488, 490 may instead be used in a predictive method that analyzes past results and modifies pace to pace timing to achieve desirable CRT in subsequent pace therapy delivery.

As indicated by the various arrows, the example may switch from one mode to another. For example, an atrial triggered mode 450 may be in use, however, upon loss of the atrial signal (caused by posture change, arrhythmia, or unknown cause) may trigger switching to use of an “other” signal in block 470, or to use of a predictive mode as indicated at 460. In several examples, a preference for cooperative modes may be in place, with switching to mode 480 performed only after other modes 450, 460, 470 are shown unreliable or ineffective. In other examples, any of modes 450, 460, 470, 480 may be used at any time simply based on which is deemed to be most reliable and/or to provide the preferred quality of CRT.

In addition, within the mode types, there may be multiple specific mode implementations such that a method or device can switch between modes of the same type. For example, an atrial triggered mode type 450 may include a first mode using the P-wave 452, and a second mode using a mechanical signal 454 such as a heart sound, and may further include a hybrid mode using each of 452, 454, if desired. If the cardiac electrical signal changes suddenly making the P-wave reliant mode 452 unusable, a mechanical atrial triggered mode 454 may still be available.

The assessment of different pacing modes, and switching between modes, may encompass the activation or deactivation of sensors and sensing capabilities specific to different modes. For example, an SICD or SCM may have multiple sensing channels and/or sense vectors that better target (using filtering or spatial differences) ventricular or atrial electrical signals. When a pacing mode relying on an electrical atrial signal is selected, the sense channel and/or sense vector best for atrial sensing may be activated; when a different pacing mode is selected, that same channel or vector may be deactivated to save power. A mechanical or optical sensor used in certain pacing modes may be deactivated when the relevant mode is not selected or under assessment.

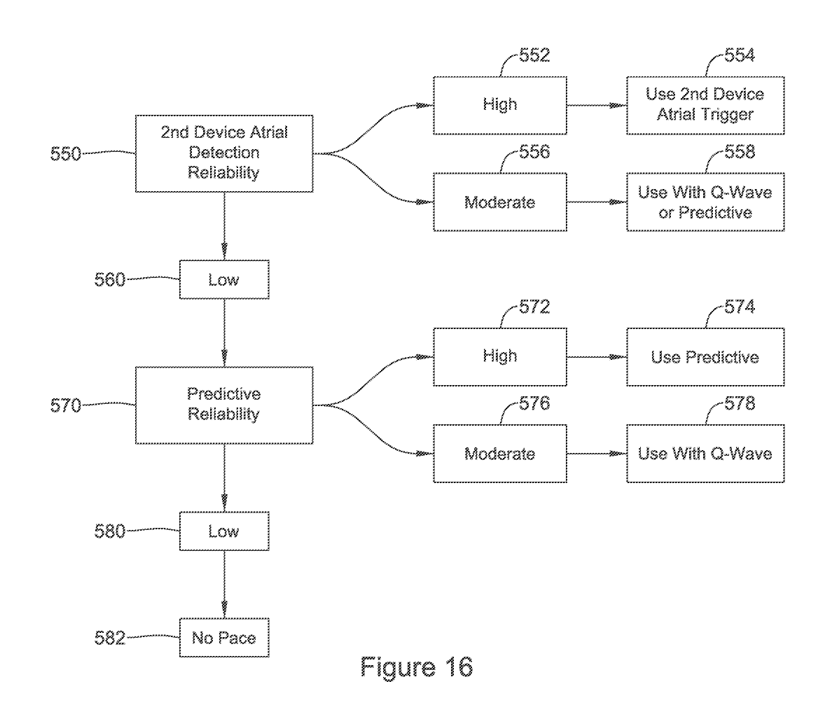

FIGS. 15-16 show illustrative examples of selecting a pacing mode. Each of FIGS. 15-16 reflect an ordered analysis in which pacing modes are ordered for analysis from more to less preferred. In FIG. 15, the method/device begins by assessment of a first mode of pacing for CRT, as indicated at 500, looking at reliability of that mode. As explained above, “reliability” in this context indicates analysis of the criteria used by the pacing mode to determine when (and/or whether) to deliver pace pulses. If the reliability is high—that is above a threshold, then the first pacing mode is selected and implemented as indicated at 502 without necessarily looking at other modes.

As used herein, an IMD that “implements” a mode of pacing may do so in several ways. In some examples, an extracardiac IMD, such as an SICD or SCM, may implement a mode of pacing by adopting a set of rules for issuing communications to an LCP that either cause the LCP to deliver commanded or requested pacing therapy, or that cause an LCP to use or adjust a particular approach to timing pacing therapy, such as by changing an interval between pace therapy deliveries. In some examples an extracardiac IMB such as an SICD or SCM may implement a mode of pacing by requesting the LCP use its own sensing circuitry, such as a sensing circuit for electrical signals, or a transducer for pressure, motion, or sound signals, or other physiological phenomena, to use for pacing timing control. Thus an IMB, such as an SICD or SCM, may implement a pacing mode without actually being the device to deliver the pace therapy.

If a threshold for the first pacing mode is not met, the method/device turns to assessing a second mode, as indicated at 510. If the second mode has reliability that meets a threshold, then the second mode may be selected and implemented as indicated at 512. A third mode is next assessed at 520, again, if the reliability exceeds a threshold, the third mode may be selected and implemented as indicated at 522.

Additional modes may be assessed in other examples (or, in the alternative, only two modes may be reviewed, if desired). If no thresholds are exceeded to terminate the analysis at any of 502, 512, or 522, the system may simply select the best of the three modes, as indicated at 524 using the assessed reliabilities. Alternatively, assuming a prior selected mode has not shown failures, the system may elect to stay with the prior selected mode, as indicated at 526. In a still further alternative, the system may elect to stop pacing as indicated at 528, if none of the criteria for pacing control are deemed acceptable.

FIG. 16 shows a more specific example of the general method of FIG. 15. The example is not intended to be limiting and is instead provided for better understanding of the concepts. In this example, a system is configured to use three pacing modes including:
A first mode of operation using a 2^{nd} Device Atrial Trigger 554—that is, a mode relying an atrial trigger by a second device to time CRT pacing (FIG. 5),
A second mode of operation using a Predictive trigger 570, relying on retrospective analysis of sensed response to a previous CRT pace to determine timing adjustments (FIGS. 7/8), and
A third mode of operation using a Q-Wave detection mode (FIG. 6).

The example is operable in a system having a first device for CRT pacing delivery in the form of an LCP located in the left ventricle. In this example, the first pacing mode is analyzed at 550 by reviewing the reliability of atrial event detection by a second device such as a second LCP or an extracardiac device (SICD or SCM). The atrial event detection may refer to electrical or mechanical detection or a hybrid thereof. If the assessment at 550 finds high reliability as indicated at 552, then the system implements a mode of operation using the first pacing mode, relying on a 2^{nd} Device Atrial Trigger, as indicated at 554.

If the assessment at 550 finds a moderate reliability 556, neither high nor low, then a combined approach is called upon as indicated at 558. In a combined approach, the first mode of operation 550 may be combined with a second mode of operation (Predictive) or third mode of operation (Q-wave detection). In combined operation, the second mode may be used either as a back-up trigger (Q-wave detection) or the second mode may be used for tailoring or optimizing the first mode (predictive).

If the assessment at 550 finds low reliability 560, this may indicate little utility for the first mode of operation at a given point in time, and so a next mode is analyzed as indicated at 570. Here reliability of a predictive mode is analyzed at 570. For example, one predictive mode (such as modes of pacing operation disclosed in U.S. Provisional Patent Application Ser. Nos. 62/378,880 and/or 62/378,866) or a plurality of predictive modes may be assessed. If the mode, or one of the modes of pacing in a class of pacing modes, has high reliability as indicated at 572, then that mode is selected and implemented standing alone as shown at 574. If instead a moderate reliability is found 576, then a combined mode may be implemented as indicated at 578, using the predictive mode alongside a triggering mode such as Q-wave detection, where the detection mode may be used to trigger pacing if such detection precedes expiration of a pace-pace interval defined by the predictive mode, if desired.

Finally, if the reliability of the predictive mode(s) assessed at 570 is low, as indicated at 580, then pacing may simply be withheld, as indicated at 582. As an alternative to pacing being terminated, an autonomous LCP mode may be triggered instead, with block 582 instead interpreted as no cooperative pacing mode.

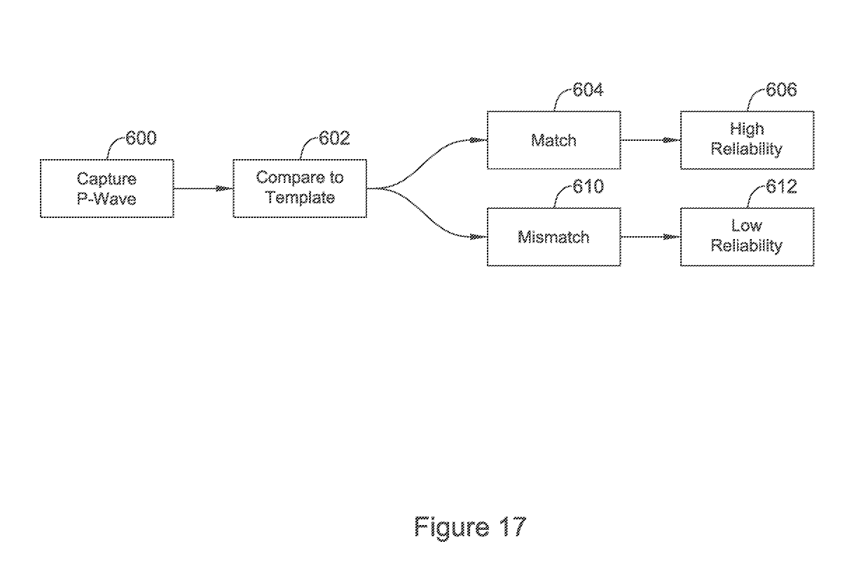

FIGS. 17-22 show in block flow form a number of examples for assessing pacing mode reliability. This set of examples is not intended to be exhaustive; other methods or criteria may be used instead.

FIG. 17 shows a first example. Here, the P-wave is captured for example by an extracardiac device such as an SICD or EGM, as indicated at 600. This may include setting a window following a prior cardiac cycle, such as by timing from a QRS complex, an R-wave, a prior P-wave, or a T-wave, and identifying a period of time in which the P-wave is expected to occur, then finding a peak in the cardiac electrical signal in the desired window that exceeds a threshold, and presuming the peak to represent a P-wave. The captured P-wave from block 600 is then compared to a template, as indicated at 602 by, for example, difference of area analysis, correlation waveform analysis, principal components analysis, wavelet transform, or by looking at features such as width, height, and polarity. If the captured P-wave matches the template, as indicated to 604, this suggests high reliability for a method that uses atrial event information such as the P-wave for triggering or predicting pace timing, as indicated at 606. Mismatch, as indicated at 610, suggests low reliability 612 for such methods, as it may be that the actual P-wave is not being detected accurately, or that the P-wave is no longer reliably occurring due to arrhythmia, or other issues such as the P-wave having unusual shape due to the effects of a partial or rate-induced heart block, for example.

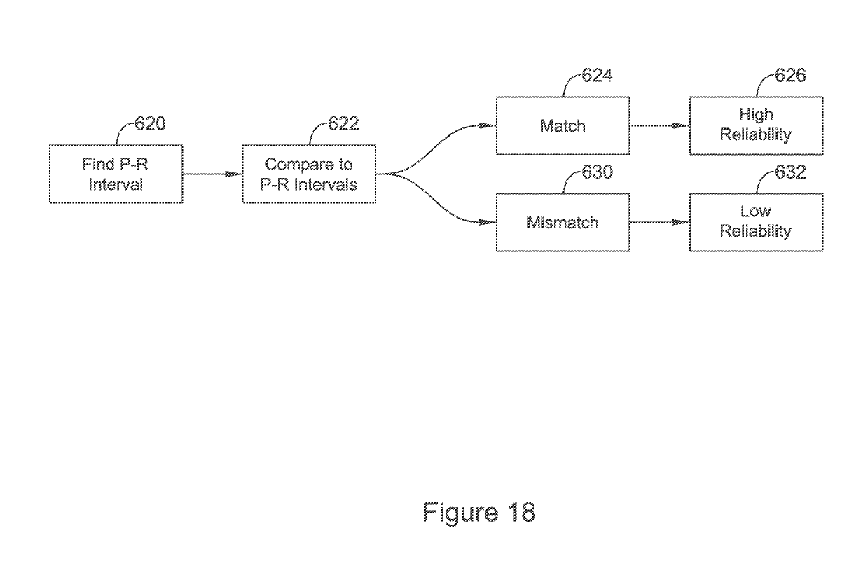

FIG. 18 shows another example. Here, the interval from the P-wave to an R-wave is measured, as indicated at 620, using again P-wave capture or detection as described above relative to block 600 in FIG. 17. The interval may be captured in real time as the signal comes in or as part of a retrospective analysis looking back at one or more cardiac cycles. The captured interval 620 is then compared to stored values of P-R intervals, as indicated at 622. The stored values may be actual measured values, or theoretical expected values, and may be scaled relative to the R-R interval to account for P-R interval reduction with increased heart rate. The stored intervals may be recently sensed intervals from other cardiac cycles associated with R-R intervals similar to the R-R interval associated with the cardiac cycle under analysis, such as being within 10% margin (plus/minus) of the cardiac cycle under analysis. If the comparison at 622 finds a match 624, this may again indicate high reliability of methods that rely on P-wave capture or detection. Mismatch 630 may again indicate low reliability 632 as suggesting influence of a heart block or arrhythmia or malsensing of the P-wave, among other possible issues.

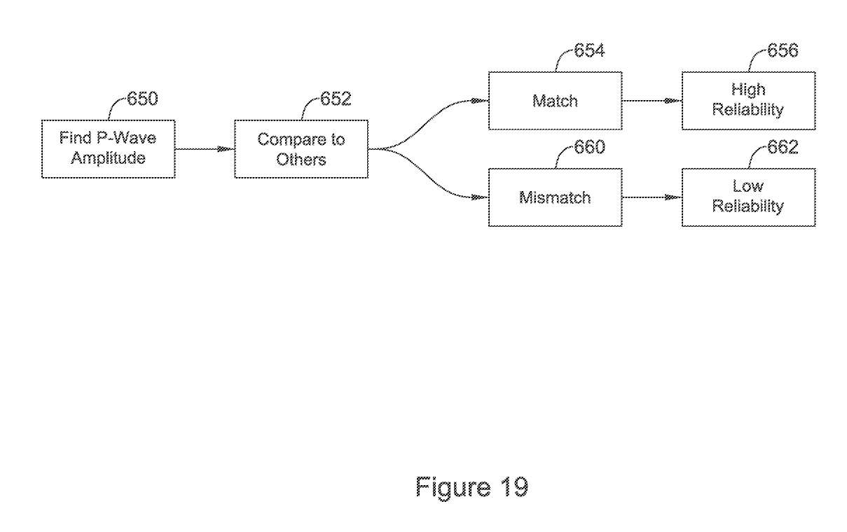

FIG. 19 shows another example. Here, the P-wave is sensed at 650 and its amplitude determined. The P-wave amplitude from 650 is compared at 652 to other P-wave amplitudes, such as a stored value or the values of close-in-time P-wave, such as the prior P-wave or an average of some quantity of prior P-waves. In one example, the other P-waves may be limited to P-waves associated with cardiac cycles having a similar R-R interval (such as within about 10% margin, plus or minus) relative to a preceding, or following, cardiac cycle. A match 654 suggests high reliability 656 for a P-wave detection method, while mismatch 660 suggests low reliability 662.

The examples of FIGS. 17-19 have been explained in the context of electrically sensing the P-wave in the cardiac electrical signal. Analogous methods may be used with heart sounds sensors or accelerometer/motion detectors instead, including capturing a representation of a mechanical signal for comparison to a template (FIG. 17), determining timing relative to other mechanical or electrical fiducials (FIG. 18), or looking at a single feature such as amplitude (FIG. 19).

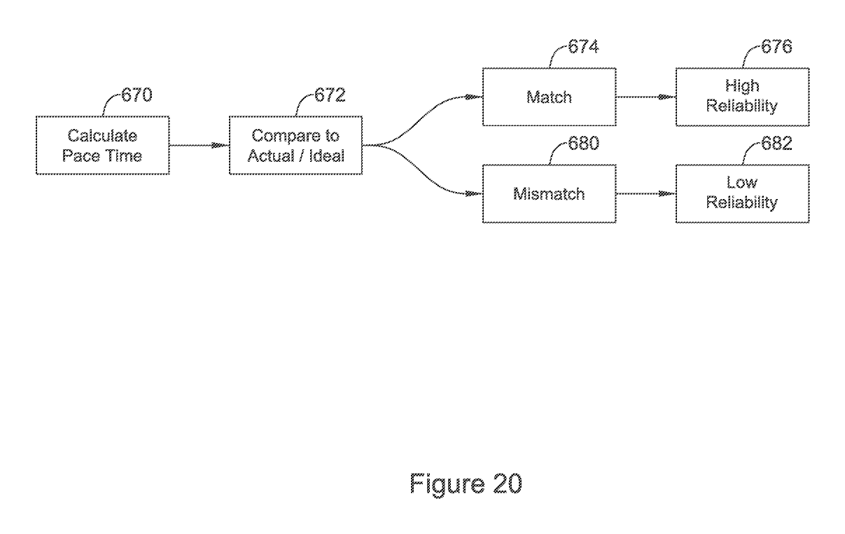

FIG. 20 shows another example. Here, the time at which a particular mode of CRT pacing (a mode not actually in use at a given time) would call for pace therapy delivery is calculated at block 670. The time from block 670 is then compared to an idealized or model pace time 672, which may be calculated using a formula or clinical assumption relative to a fiducial identified in the cardiac electrical signal. For example, the method may compare the time from block 670 to a point in time that is 120 milliseconds after the P-wave (a point in time used by some physicians in planning CRT pacing with conventional systems). Other reference points may be used. For example, a testing sequence may be performed to determine an optimal pace time relative to the P-wave, QRS complex, or R-wave peak that generates fusion beats by delivering pace therapies at various intervals relative to the selected fiducial until fusion is identified, with the interval that yields fusion stored as an “ideal” interval for use in block 672 and/or elsewhere in an algorithm. Once again, a match 674 suggests high reliability 676, while a mismatch 680 suggests low reliability 682.

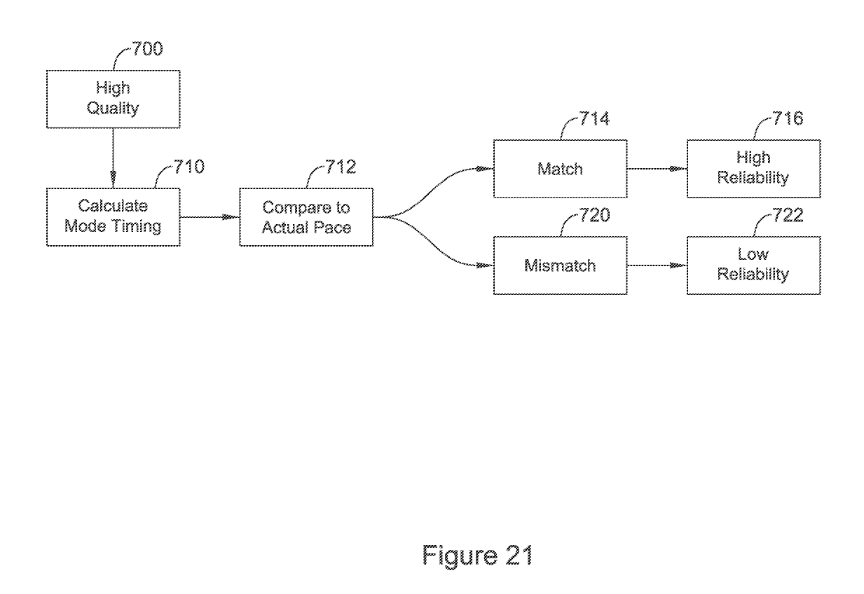

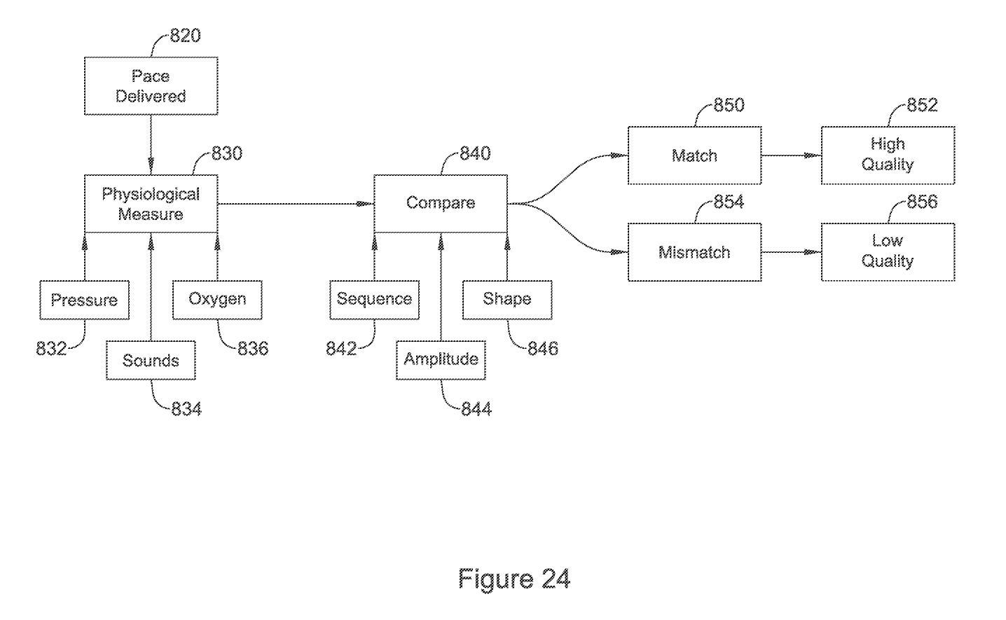

FIG. 21 shows another example. Here, an assessment is first made as to the quality of the outcome from CRT pacing, as indicated at 700. Examples of such quality assessments are shown in FIGS. 23-24, below. Beginning at block 700, a cardiac cycle is analyzed in which the pace outcome is high quality (a fusion beat may have occurred, for example). The mode being assessed is then simulated to generate mode timing 710—the time at which the mode under analysis would have called for pacing therapy delivery. The mode timing is then compared to the actual pace timing 712 that yielded the high quality outcome 700. A match 714 shows that the mode under review would most likely have also generated a high quality outcome using its input criteria, and so the mode is associated with high reliability 716. On the other hand, mismatch 720 suggests the mode under analysis would not have yielded a high quality outcome for the cardiac cycle under analysis, and is therefore associated with low reliability 722.

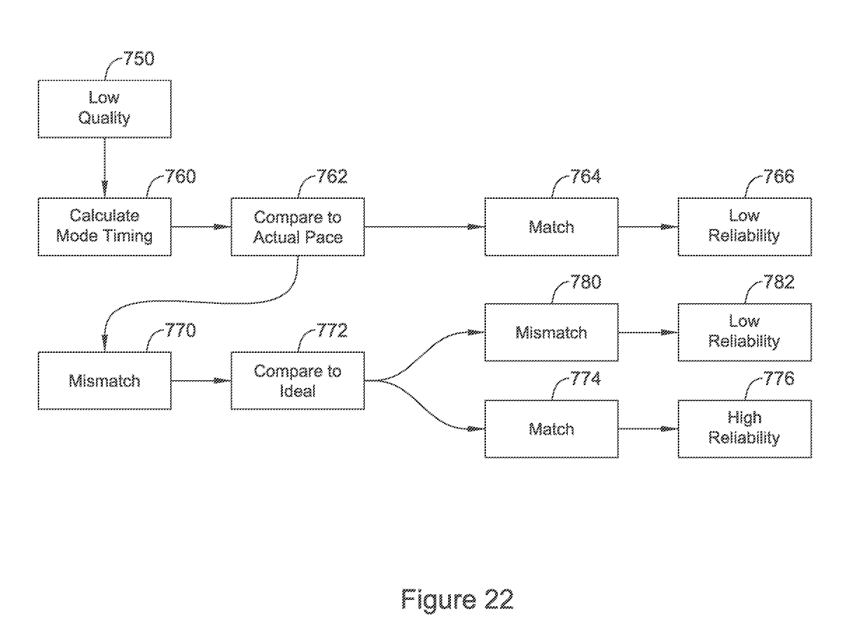

FIG. 22 shows another example. Here, a low quality outcome 750 has occurred for a given cardiac cycle. Again the mode timing is calculated at 760, and compared to the actual pace time at 762. A match 764 indicates that the mode under review would have yielded the same low quality outcome, and so a low reliability is found at 766.

Mismatch 770 may or may not be a good thing. A low quality outcome 750 may occur due to pace delivery too early or too late. Therefore the method next determines when the ideal pace would have occurred, at 772, again using a timing fiducial generated from the cardiac electrical or mechanical signal(s). A match 774 suggests high reliability 776, while mismatch 780 would suggest low reliability 782.

In some examples, the comparison to ideal at 772 may be replaced by analysis of whether the pace therapy actually delivered was too early or too late, using methods indicated in U.S. Provisional Patent Application Ser. No. 62/378,866, the disclosure of which is incorporated herein by reference, which suggest that analysis using templates or rules, applied to a QRS after a CRT pace delivery, may find the QRS complex indicative of an LV captured beat (meaning the pace therapy was too early in the cardiac cycle relative to the P-wave or QRS complex), or indicative of a native or intrinsic beat (meaning pace therapy came too late to affect the cardiac cycle). Then the comparison to ideal at 772 may instead simply determine, based on the QRS analysis, whether the actual pace was too early or too late, and then determines if the mode timing would have been different in the right direction (if the actual pace was too early and the mode timing would have issued the pace therapy later in time, this is a change in the right direction). A change in the right direction would be treated as a match 774 associated with high reliability, 776, and a change which does not would be treated as a mismatch associated with low reliability 782.

In some examples, a plurality of analyses as in FIGS. 21-22 may be performed for each of two or modes of CRT pacing to generate a statistical understanding of the separate modes. For example, given a set of 20 paced beats, 12 of which yielded fusion outcomes and 8 of which did not, the method or device may simulate the analysis of each of the modes under consideration repeatedly for the 20 paced beats. Then the likelihood of good and poor outcomes with each of the analyzed methods may be assessed. 20 beats or cardiac cycles is merely an illustrative quantity; higher or lower numbers may be used. Rather than a set of 20, a system may track data for each of a set of most recent good pacing outcomes and a set of most recent poor pacing outcomes, to allow assessment of both positive and negative likelihoods. In another example, only paced beats having good outcomes may be analyzed, avoiding any reliance on assumed ideal pace timing.

FIGS. 23-24 show illustrative examples for assessing quality of delivered pace therapy. Starting with FIG. 23, a pace therapy is delivered at 800, and the QRS complex following pace delivery is captured, as indicated at 802. Characteristics of the QRS complex, or the actual signal thereof, can then be compared to a set of rules or a template, as indicated at 804. The comparison at 804 may resemble the capture verification techniques discussed in U.S. Provisional Patent Application Ser. No. 62/378,866, the disclosure of which is incorporated herein by reference. A match at 806 suggests a high quality pacing outcome 808, such as a fusion beat. Mismatch 810 suggests low quality pacing outcome 812, such as a lack of fusion.

FIG. 24 illustrates another example, this time using physiological information rather than the cardiac electrical signal. A pace is delivered 820 and a physiological measurement is performed as indicated at 830. The physiological measure may take the form of one or more of a pressure measurement 832, capture of heart sounds 834, or monitoring of blood oxygenation 836 to observe, for example, the strength of pulsatile flow; alternatives not shown may track the ventricular volume using impedance, analyze the frequency content of one or more measures (such as heard sounds), or any other suitable physiological outcomes indicative of whether CRT is successful on a beat to beat basis. Various measures may be combined together with other physiological measures or may be used in combination with the cardiac electrical signal. The physiological measure 830 is then compared at 840 to a desired outcome. The desired outcome may include a sequence 842 of measurements or events, amplitude 844 of one or more signals (or change of amplitude), and/or an assessment of shape 846, and/or combinations thereof. A match 850 suggests a high quality outcome 852, and mismatch 854 suggests a low quality outcome 856.

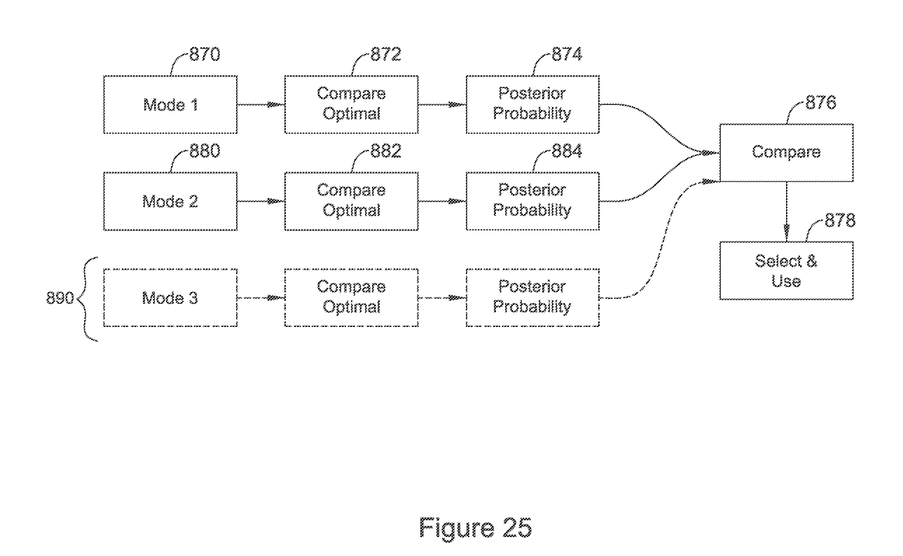

FIG. 25 shows an illustrative example using posterior probability. In this example, a number of parallel analyses may occur. In one track, a first mode of CRT pacing is analyzed by looking at data for a plurality of cardiac cycles stored in a memory to simulate application of the first mode 870. The results of the simulations are then compared to an optimal outcome, as indicated at 872.

For example, criteria used by a first mode may include atrial signal data such as detection of a P-wave using selected parameters to trigger pacing by an LCP. A simulation would subject the data captured for a cardiac cycle to the analysis of the first mode and determine when the first mode would trigger pacing. In some examples, an optimal outcome 872 would also be calculated for the cardiac cycle by reference to the total signal of the cardiac cycle to calculate an optimal time at which pacing would be triggered. For example, the optimal time for pacing may be a set duration prior to the R-wave or QRS complex. The comparison at 872 can yield one or more differences for a plurality of cardiac cycles, from which a posterior probability of well-timed pacing can be determined. A credible interval may be defined to allow an error, for example, of plus-minus a certain margin around the optimal time, such as a plus minus 10 to 20 milliseconds interval. A posterior probability of the mode 870 yielding desirable CRT outcomes based on available data is then determined. The process is repeated with a second mode 880 undergoing simulations and comparison to optimal at 882 to provide another posterior probability at 884. The posterior probabilities are compared at 876 and the mode deemed most likely to provide the desired results is selected and implemented at 878. A third mode may be included in the analysis as well as indicated at 890.

In another example, the optimal outcome, rather than being idealized, may instead be determined by analyzing paced beats using heart sounds or morphology, for example, to identify successful CRT pacing that yields fusion beats. The paced beats can be set into two categories: those with desirable CRT pacing results, and those lacking desirable CRT pacing results. To compare to optimal at blocks 872, 882, the simulations area run using the data from the paced beats in each category to provide statistical inputs for the posterior probability analysis. Again a credible window, such as plus/minus 10 to 30 milliseconds around the actual paces (both successful and not) may be defined for purposes of the analysis.

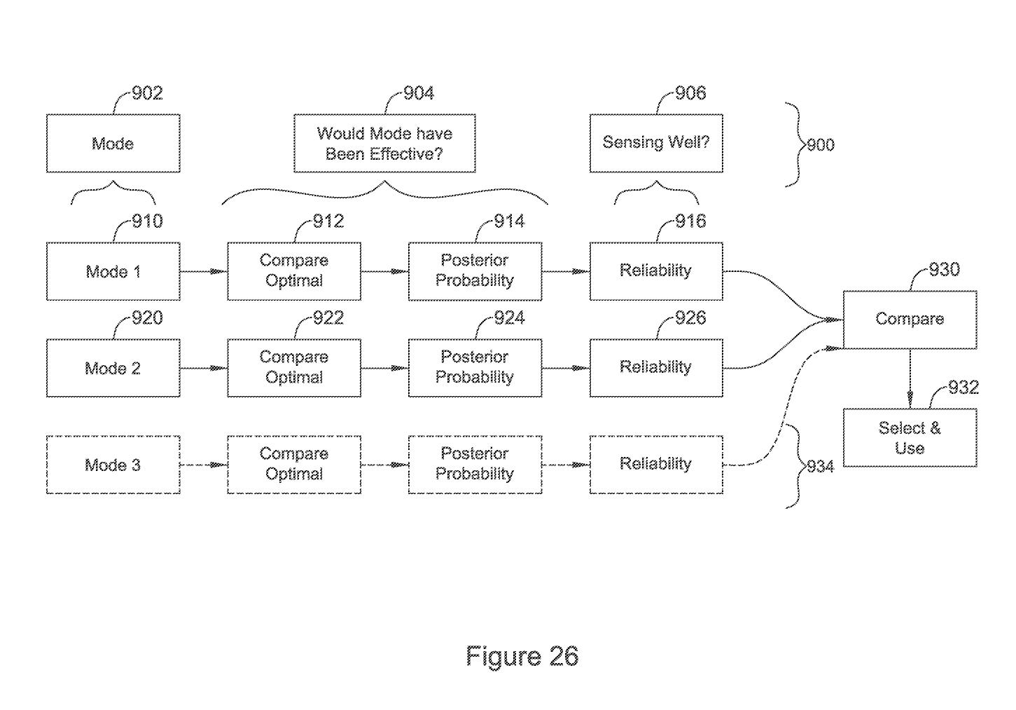

FIG. 26 shows an example incorporating both posterior probability and mode reliability. The approach is summarized at 900 as including assessment of two or more modes of CRT pacing, determining whether a given mode would have been effective 904 if applied to prior cardiac cycles, and asking as well whether the mode under analysis relies on criteria which are currently being sensed well, as indicated at 906.

More particularly, as indicated for a first mode of CRT pacing 910, a plurality of simulations are run to allow comparison to an optimal pace timing 912. The optimal pace timing may be determined analytically for individual cardiac cycles or may be based on actual cardiac responses to delivered therapies, as discussed above. The posterior probability(s) of beneficial and/or negative outcomes with the mode are calculated at 914. Next, the current reliability is assessed at 916 using, for example, metrics relevant to the given mode (that is, if a P-wave detection based mode is under analysis, then P-wave related metrics may be assessed such as amplitude, shape and stability; other relevant metrics are discussed above). Similar assessment occurs for a second mode 920 including simulation and comparison to theoretical or real-world optimal timing 922, calculation of posterior probability 924, and assessment of reliability.

A combination of the posterior probability and current reliability are then compared across multiple modes, as indicated at 930. In some examples, a single mode is then selected and implemented as indicated at 932. Alternatively, outcomes of multiple selected modes may be combined together if desired. In addition to the first and second modes 910, 920, additional modes such as at 934 may also be analyzed.

Some examples may use a Bayesian statistical model to implement the method in FIG. 26. In other examples, a scoring approach can be used where in the posterior probability and reliability metrics are converted to first and second sub-scores and added or multiplied together to yield a final score. In particular, the reliability metrics may come from very different inputs across different modes of CRT pacing control, such that a conversion table may be useful to allow equivalency to be drawn, for example, between a reliability assessment of an approach relying on a mechanical sensor and reliability of an approach using a retrospective analysis of the QRS morphology after a CRT pace therapy is delivered.

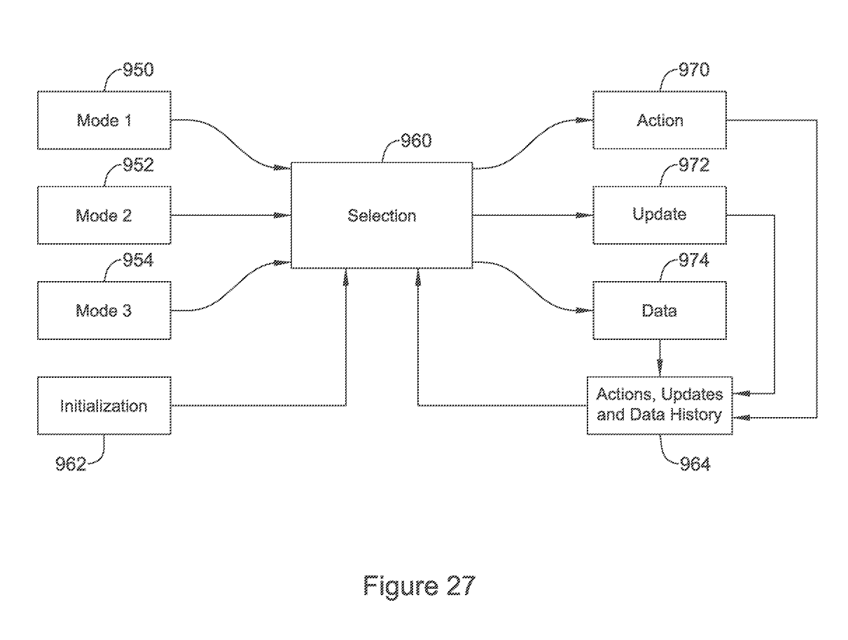

FIG. 27 shows another illustrative example. In FIG. 27, a plurality of modes 950, 952, 954 for controlling pacing are operable within a system; the different modes may use different analyses to determine timing for CRT therapy such as shown in various examples above. As indicated by the dashed lines, in some examples “Mode 3” 954 can be omitted.

In an example, each mode 950, 952, 954 is executed on an ongoing basis, with a selection block 960 used to choose which modes 950, 952, 954 can be used to drive various purposes, including taking action 970, which may include delivering a pace therapy (from the perspective of an LCP) or commanding or requesting delivery of a pace therapy (from the perspective of a second device such as an extracardiac device for example), updating as indicated at 972 (which may modify a sensing criteria used by one of the modes 950, 952, or change a selected mode for action 970, for example), or for data use as indicated at 974 (which may include storing posterior probability information or current reliability information for one or more modes 950, 952, 954 which may itself include data related to when pace therapy was in fact delivered, the result of delivered pace therapy, and what timing would have been applied by the different modes). For example, data stored at 974 may be used as illustrated above to determine which, if any, of the different modes would be able to generate fusion, and/or how likely fusion is to occur if each mode is used.

In making the selection 960, initialization data 962 may be used to determine which mode 950, 952, 954 has outputs that are directed to which use 970, 972, 974. The selection block 960 may also reference updates, data, and action history 964 to tailor its operation in light of post-initialization activity. For example, initialization 962 may indicate that a certain mode 950/952/954 is to be the default mode, with one or more other modes as a backup as suggested above, such that selection 960 uses the initial default mode except if otherwise indicated by the updates or data 964.

In an illustration, all or a plurality of the modes 950, 952, 954 may be operating on incoming data at any given time, with the selection block 960 determining which mode is to be used to trigger action 970, as well as designating updates 972 to be used for updating ongoing selection and/or sensing activity of the modes 950, 952, while also storing data 974 for use in subsequent assessment of reliability and/or probability of success with any given mode. For example, the updates 972 may comprise adjusting a sensing window used to identify P-waves, or a sensing threshold applied to identify P-waves.

The updates 972 may also be used to adjust therapy. For example, a first mode 950 may be a CRT pacing mode that uses an extracardiac device such as an SICD or SCM to detect atrial events (P-waves) and command or request pacing by an LCP. In such a mode 950, there may be an adjustable delay effected by the extracardiac device or the LCP before pace therapy is actually delivered. A second mode may use a morphology analysis to determine whether a QRS complex following pace therapy delivery illustrates desired fusion response from the patient's heart. The second mode may determine that therapy came too early, or too late, based on setting of the first mode and, if so, an update 972 may include the second mode 952 adjusting the adjustable delay of the first mode 950.

In an alternative, referring back to FIG. 13, all but one of the modes may be disabled during a given time period as pacing is delivered in block 400. Upon an event, such as expiration of a quantity of pace therapies 402 or 404, or after elapsed time 406, or upon occurrence of a failure or exception 408, reliability is assessed at 410 using all modes 412, or just the current mode 414, or using a sequence of different modes 416, to look at reliability 410. In some examples, the reliability assessment 410 may be supplemented by the use of posterior probability calculations (FIG. 25) of how well one or more individual modes would have worked had they been in use, for example by using stored data relating to prior pace therapies, the effects of such pace therapies, and data inputs that would have been used for alternative modes (FIG. 26).

For example, some systems may use retrospective assessment of a plurality of modes of pacing operation to determine posterior probability and/or reliability in a manner that relies on stored data for a period of time, such as 10 seconds up to 10 minutes of time. By so doing, the need to continuously assess multiple modes can be avoided, saving on the energy burden of extensive and repeated computation. Alternatively, a system may be designed such that the computational energy burden of continuous or real time assessment of multiple pacing modes is of little actual impact to device life, making the continuous approach more useful.

A series of illustrative and non-limiting examples follows. These examples are provided for further illumination and is should be understood that other embodiments using other combinations of features are also contemplated.

A first illustrative and non-limiting example takes the form of an implantable medical device (IMD) configured for use as part of a cardiac therapy system comprising a leadless cardiac pacemaker (LCP) for delivering cardiac resynchronization therapy (CRT) and the IMD, the IMD comprising: a plurality of electrodes for sensing cardiac signals; communication circuitry for communicating with the LCP; and operational circuitry configured to receive sensed cardiac signals from the plurality of electrodes and analyze cardiac activity. In the first illustrative and non-limiting example, the operational circuitry comprises first mode means for implementing a first mode of CRT pacing in cooperation with the LCP, such as stored instruction sets or dedicated circuitry or a combination thereof to operate using a select one of the pacing modes 450, 460, 470, 480 in FIG. 14, or modes 500, 510, 520 in FIG. 15, modes 550, 570 in FIG. 16, or modes 870, 880 in FIG. 25, or modes 910, 920 in FIG. 26, or modes 950, 952, 954 in FIG. 27; such modes of operation are further explained in FIGS. 5-12. In the first illustrative and non-limiting example, the operational circuitry also comprises second mode means for implementing a second mode of CRT pacing in cooperation with the LCP, such as stored instruction sets or dedicated circuitry or a combination thereof to operate using another selected one of the pacing modes 450, 460, 470, 480 in FIG. 14, or modes 500, 510, 520 in FIG. 15, or modes 870, 880 in FIG. 25, or modes 910, 920 in FIG. 26, or modes 950, 952, 954 in FIG. 27; such modes of operation are further explained in FIGS. 5-12. In the first illustrative and non-limiting example, the operational circuitry further includes selection means for selecting between the first and second mode means, such selection means being, for example, stored instruction sets or dedicated circuitry or a combination thereof to operate as shown and described relative to the combination of reliability assessment 410 and switching 420 in FIG. 13, or through tiered assessment as in FIG. 15's selection from modes 1, 2 and 3 at 500, 510, 520, or by FIG. 16's tiered analysis of modes at 550, 570, or by the combination of comparison 876 and selection/use 878 in FIG. 25, or by the combinations of comparison 930 and selection/use 932 in FIG. 26, or with selection block 960 in FIG. 27, for example.

A second illustrative and non-limiting example takes the form of an IMD as in the first illustrative and non-limiting example wherein the selection means is configured to assess reliability of the first mode means and the second mode means through its analysis of the sensed cardiac signals. Such assessment of reliability are illustrated relative to block 410 of FIG. 13, blocks 500, 510, 520 of FIG. 15, blocks 550, 570 of FIG. 16, at blocks 906, 916, 926 in FIG. 26, all of which may be integrated into block 960 of FIG. 27. Moreover, various assessments of reliability are shown in FIGS. 17-22.

A third illustrative and non-limiting example takes the form of an IMD as in the second illustrative and non-limiting example wherein the first mode means uses detection of atrial events to implement CRT pacing (such is shown in FIGS. 5, 9 and 10 as well as being summarized in FIG. 14 at 450), and wherein the selection means is configured to assess reliability of the first mode means by analyzing one or more of the shape and amplitude of one or more atrial signals (FIGS. 17 and 19 show examples of analysis of the atrial signals to determine reliability).

A fourth illustrative and non-limiting example takes the form of an IMD as in the second illustrative and non-limiting example further comprising interval means for determining intervals in the sensed cardiac signals including at least an interval from a P-wave to an R-wave of the same cardiac cycle (interval identification means may comprise dedicated circuitry and/or stored instruction sets for operation by a controller, or combinations thereof, as indicated at block 620 in FIG. 18); wherein the selection means is configured to assess reliability of the first mode means by determine whether intervals from P-wave to R-wave in a selected set of cardiac cycles are similar to one another (as indicated in the remainder of FIG. 18). A fifth illustrative and non-limiting example takes the form of an IMD as in the fourth illustrative and non-limiting example wherein the set of cardiac cycles is selected such that each cardiac cycle has a similar cardiac cycle length (as explained relative to block 622 in FIG. 18).

A sixth illustrative and non-limiting example takes the form of an IMD as in any of the first to fifth illustrative and non-limiting examples, wherein the first mode means is operable by the IMD using the following: atrial event means for detecting an atrial event; and issuance means for determining to issue a communication to the LCP in response to detection of an atrial event by the atrial event means. Such a mode is illustrated in FIG. 5, with atrial event means taking the form of dedicated circuitry and/or stored instruction sets, or combinations thereof, as indicated at block 250 in FIG. 5, and communication means taking the form of dedicated circuitry and/or stored instruction sets, or combinations thereof, as indicated at block 252 in FIG. 5.

A seventh illustrative and non-limiting example takes the form of an IMD as in any of the first to sixth illustrative and non-limiting examples, wherein the selection means is configured to assess reliability of a selected one of the first or second mode means using: quality means for assessing the quality of outcome for the paced cardiac cycles assessed by the timing means; timing means to determine a timing difference between when a pace therapy was delivered by the LCP and when a pace therapy would have been delivered if using the selected one of the first or second mode means to implement CRT on the LCP for a plurality of paced cardiac cycles; and comparison means to compare the timing differences found by the timing means to the quality of outcomes determined by the quality means. FIG. 21 shows such quality means at 700, timing means at 710, and comparison means at 712, wherein each may take the form of dedicated circuitry, stored instruction sets, or combinations thereof, to perform as indicated in the figure and described therewith.

An eighth illustrative and non-limiting example takes the form of an IMD as in any of the first to sixth illustrative and non-limiting examples, wherein the selection means comprises quality means for analyzing a set of cardiac cycles paced by the LCP for CRT in accordance with timing information from the first mode means to determine the quality of outcomes of the set of cardiac cycles, and the selection means is configured to assess the reliability of the first mode means using results from the quality means. For example, FIG. 23 illustrates the pace delivery at 800, and analysis at 802/804 to generate quality outcomes at 808, 812. In addition, FIG. 23 illustrates that pace delivery 820 can be used to support the capture of physiological measures 830 of outcomes for comparison 840 to relevant metrics, yielding determination of quality at 852 or 856. Finally, FIG. 13 illustrates that the currently used mode 414 can be assessed as part of a reliability assessment 410.

A ninth illustrative and non-limiting example takes the form of an IMD as in any of the seventh or eighth illustrative and non-limiting examples, wherein the quality means is configured to use a template for a fusion beat to determine the quality of outcomes of the paced cardiac cycles. The use of such templates for quality assessment is discussed at a number of places including, for example, in block 804 of FIG. 23.

A tenth illustrative and non-limiting example takes the form of an IMD as in any of the seventh or eighth illustrative and non-limiting examples, wherein the quality means is configured to use a physiological measure to determine the quality of outcomes of the paced cardiac cycles. The use of these physiological measures is illustrated in FIG. 24. An eleventh illustrative and non-limiting example takes the form of an IMD as in the tenth illustrative and non-limiting example, wherein the physiological measure comprises heart sounds, with FIG. 24 specifically calling out heart sounds at block 834.

A twelfth illustrative and non-limiting example takes the form of an IMD as in the first illustrative and non-limiting example, wherein the selector means comprises: probability means for determining a probability of accuracy of pace timing calculations for each of the first mode means and second mode means for at least one cardiac cycle; wherein the selector means is configured to use the probability of accuracy of pace timing to select from the at least first mode means and second mode means. FIGS. 25 and 26 illustrate that multiple modes would be analyzed by comparison to “optimal” or at least good pace timing to generate a probability of accuracy of pace timing.

A thirteenth illustrative and non-limiting example takes the form of an IMD as in the twelfth illustrative and non-limiting example, wherein the selector means further comprises: P-wave analytic means to calculate one or more analytics of one or more P-waves in one or more cardiac cycles; and reliability means to calculate a reliability for at least the first mode means based on data from the P-wave analytic means; wherein the selector means is configured to use the reliability in combination with the probability of accuracy of pace timing of at least the first mode means. FIG. 26 illustrates a combination of the probability of accuracy 904, 914, 924, and reliability 906, 916, 926 for the selector means (as indicated above, the combination of 930 and 932), with the reliability assessments including, as shown in FIGS. 17-19, P-wave analytics.

A fourteenth illustrative and non-limiting example takes the form of an IMD as in any of the first to thirteenth illustrative and non-limiting examples, wherein the first mode means relies on detection of an atrial event to cause pace therapy in the same cardiac cycle as the atrial event, and the second mode means is operable by analyzing one or more cardiac cycles as or after they occur to determine when a pace therapy should be delivered for a subsequent cardiac cycle. FIG. 16 show a specific example in which a first mode uses the atrial detection as indicated at 550, and a second mode uses a predictive approach as indicated at 570 that operates by analyzing one or more cardiac cycles as or after they occur to determine when a pace therapy should be delivered (hence, predictive) in a subsequent cardiac cycle.

A fifteenth illustrative and non-limiting example takes the form of an IMD as in any of the first to thirteenth illustrative and non-limiting examples, wherein: the operational circuitry further comprises third mode means configured to use detection of a septal electrical signal event of a patient's heart to cause pace therapy in the same cardiac cycle as the septal electrical signal event. A general example is in FIG. 15, with a third mode at 520/522, (noting that FIGS. 6, 11 and 12 disclose septal event detection as a trigger), and a specific example in FIG. 16, with inclusion of a septal event detection indicated at 578. Further in the fifteenth illustrative and non-limiting example, the first mode means relies on detection of an atrial event to cause pace therapy in the same cardiac cycle as the atrial event, and the second mode means is operable by analyzing one or more cardiac cycles as or after they occur to determine when a pace therapy should be delivered for a subsequent cardiac cycle. FIG. 16 show a specific example in which a first mode uses the atrial detection as indicated at 550, and a second mode uses a predictive approach as indicated at 570 that operates by analyzing one or more cardiac cycles as or after they occur to determine when a pace therapy should be delivered (hence, predictive) in a subsequent cardiac cycle. Also, again, FIG. 15 shows a more general example, again noting that FIGS. 5, 9 and 10 disclose the reliance on triggering from an atrial detection, and FIGS. 7 and 8 disclose post-pace data analysis and adjustments of pace timing for subsequent cardiac cycles.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic or optical disks, magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description.

The Abstract is provided to comply with 37 C.F.R. § 1.72(b), to allow the reader to quickly ascertain the nature of the technical disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. An implantable medical device (IMD) configured for use as part of a cardiac therapy system comprising a leadless cardiac pacemaker (LCP) for delivering cardiac resynchronization therapy (CRT) and the IMD, the IMD comprising:
a plurality of electrodes for sensing cardiac signals;
communication circuitry for communicating with the LCP; and
operational circuitry configured to receive sensed cardiac signals from the plurality of electrodes and analyze cardiac activity;
wherein the operational circuitry is configured to selectively implement a first mode of CRT pacing using first criteria for timing the delivery of CRT pacing by the LCP;
wherein the operational circuitry is configured to selectively implement a second mode of CRT pacing using second criteria for timing the delivery of CRT pacing by the LCP; and
wherein the operational circuitry is configured to select a mode for implementation amongst at least the first and second modes of CRT pacing and communicate to the LCP to implement the selected mode.

2. The IMD of claim 1 wherein the operational circuitry is configured to assess reliability of the at least first and second modes by analyzing data related to the first and second criteria, and to use the reliability to select a mode for implementation.

3. The IMD of claim 1 wherein the operational circuitry is configured to assess quality of a selected one of the at least first and second modes of CRT pacing to determine whether the selected one of the at least first and second modes is effectively providing CRT, and to use the quality to select a mode for implementation.

4. The IMD of claim 1 wherein the operational circuitry is configured to assess quality of a selected one of the at least first and second modes of CRT pacing to determine whether the selected one of the at least first and second modes will likely effectively provide CRT, and to use the quality to select a mode of CRT pacing for implementation.

5. The IMD of claim 1 wherein the operational circuitry is configured to operate at least the first and second modes of CRT pacing to determine timing of pacing outputs that the first and second modes would have generated for a plurality of cardiac cycles, and to compare to actual timing of pace delivery by the LCP to assess past accuracy of the at least first and second modes of CRT pacing.

6. The IMD of claim 5 wherein the operational circuitry is configured to generate one or more measures of probability related to the past accuracy of the at least first and second modes of operation.

7. An implantable medical device (IMD) configured for use as part of a cardiac therapy system comprising a leadless cardiac pacemaker (LCP) for delivering cardiac resynchronization therapy (CRT) and the IMD, the IMD comprising:
a plurality of electrodes for sensing cardiac signals;
communication circuitry for communicating with the LCP; and
operational circuitry configured to receive sensed cardiac signals from the plurality of electrodes and analyze cardiac activity;
wherein the operational circuitry is configured to selectively implement a first mode of CRT pacing using first criteria for timing the delivery of CRT pacing by the LCP;
wherein the operational circuitry is configured to selectively implement a second mode of CRT pacing using second criteria for timing the delivery of CRT paces by the LCP; and
wherein the operational circuitry is configured to:
determine probabilities of pacing for CRT at a desirable time for at least the first and second modes of CRT pacing;
determine a current reliability for each of the first and second modes of CRT pacing;
select between the first and second modes of CRT pacing using the probabilities and the current reliabilities; and
implement the selected mode of CRT pacing.

8. An IMD as in claim 7, wherein one of the first mode of CRT pacing or the second mode of CRT pacing uses atrial electrical events as first criteria, and reliability of the first mode is analyzed by observing one or more of amplitude, shape, or timing of a signal representative of the atrial electrical events.

9. An IMD as in claim 7, wherein one of the first mode of CRT pacing or the second mode of CRT pacing uses atrial mechanical events as first criteria, and reliability of the first mode is analyzed by observing one or more of amplitude, shape, or timing of a signal representative of the atrial mechanical events.

10. An IMD as in claim 7, wherein one of the first mode of CRT pacing or the second mode of CRT pacing uses septal electrical events as first criteria, and reliability of the first mode is analyzed by observing one or more of amplitude, shape, or timing of a signal representative of the septal electrical events.

11. An IMD as in claim 7, wherein one of the first mode of CRT pacing or the second mode of CRT pacing uses retrospective analysis of QRS complex shape as first criteria, and reliability of the first mode is analyzed by observing one or more of amplitude, shape, or timing of a signal representative of the QRS complexes.

12. An IMD as in claim 7, wherein one of the first mode of CRT pacing or the second mode of CRT pacing uses retrospective analysis of a plurality of electrical events in the cardiac cycle as first criteria, and reliability of the first mode is analyzed by observing one or more of amplitude, shape, or timing of a signal or signals representative of the plurality of electrical events.

13. An implantable cardiac therapy system comprising:
a leadless cardiac pacemaker (LCP) for delivering cardiac resynchronization therapy (CRT); and
an implantable medical device (IMD) comprising a plurality of electrodes for sensing cardiac signals, communication circuitry for communicating with the LCP, and operational circuitry configured to receive sensed cardiac signals from the plurality of electrodes and analyze cardiac activity;
wherein the operational circuitry is configured to selectively implement a first mode of CRT pacing using first criteria for timing the delivery of CRT pacing by the LCP;
wherein the LCP is configured to selectively implement a second mode of CRT pacing using second criteria for timing the delivery of CRT paces by the LCP; and
wherein the operational circuitry is configured to select a mode for implementation amongst at least the first and second modes of CRT pacing and communicate to the LCP to implement the selected mode.

14. The system of claim 13 wherein one of the first mode of CRT pacing or the second mode of CRT pacing uses atrial electrical events as first criteria, and reliability of the first mode is analyzed by observing one or more of amplitude, shape, or timing of a signal representative of the atrial electrical events.

15. The system of claim 13 wherein one of the first mode of CRT pacing or the second mode of CRT pacing uses atrial mechanical events as first criteria, and reliability of the first mode is analyzed by observing one or more of amplitude, shape, or timing of a signal representative of the atrial mechanical events.

16. The system of claim 13 wherein one of the first mode of CRT pacing or the second mode of CRT pacing uses septal electrical events as first criteria, and reliability of the first mode is analyzed by observing one or more of amplitude, shape, or timing of a signal representative of the septal electrical events.

17. The system of claim 13 wherein one of the first mode of CRT pacing or the second mode of CRT pacing uses retrospective analysis of QRS complex shape as first criteria, and reliability of the first mode is analyzed by observing one or more of amplitude, shape, or timing of QRS complexes.

18. The system of claim 13 wherein one of the first mode of CRT pacing or the second mode of CRT pacing uses retrospective analysis of a plurality of electrical events in the cardiac cycle as first criteria, and reliability of the first mode is analyzed by observing one or more of amplitude, shape, or timing of the plurality of electrical events.

19. The system of claim 13 wherein the operational circuitry is configured to select the first mode of CRT pacing by default, and to switch to the second mode of CRT pacing in response to a failure of the first mode of CRT pacing to generate fusion beats.

20. The system of claim 13 wherein the operational circuitry is configured to select the first mode of CRT pacing by default, and to switch to the second mode of CRT pacing in response to finding that the first mode of CRT pacing is unreliable due to changes in or absence of a signal relied upon to determine the first criteria by the first mode of CRT pacing.
